(19) United States
(12) Patent Application Publication
(10) Pub. No.: US 2026/0166032 A1
(43) Pub. Date: Jun. 18, 2026

(54) STEAROYL-COA DESATURASE 1 (SCD1) INHIBITORS FOR THE TREATMENT OF HIGH-RISK MEDULLOBLASTOMA

(71) Applicant: McMaster University, Hamilton (CA)

(72) Inventors: Sheila Singh, Dundas (CA); Chitra Venugopal, Burlington (CA); Stefan Custers, Dundas (CA)

(21) Appl. No.: 19/355,132

(22) Filed: Oct. 10, 2025

Related U.S. Application Data
(60) Provisional application No. 63/705,809, filed on Oct. 10, 2024.

(51) Int. Cl. A61K 31/501; A61K 31/167; A61K 31/428; A61K 31/444; A61K 31/445; A61K 31/498; A61K 31/4985; A61K 45/06; A61P 35/00

(52) U.S. Cl. CPC A61K 31/501; A61K 31/167; A61K 31/428; A61K 31/444; A61K 31/445; A61K 31/498; A61K 31/4985; A61K 45/06; A61P 35/00

(57) ABSTRACT

This disclosure relates to stearoyl-CoA Desaturase 1 (SCD1) inhibitors for treating medulloblastoma such as high risk medulloblastoma. For example, the high risk medulloblastoma comprises a G3 medulloblastoma, a G4 medulloblastoma, G3 medulloblastoma that is MYC amplified (MYC-driven G3-MB), or a G4 medulloblastoma that is MYC amplified (MYC-driven G4-MB).

### RELATED APPLICATIONS

The present application claims the benefit of priority of co-pending U.S. provisional patent application No. 63/705,809 filed on Oct. 10, 2024, the contents of which are incorporated herein by reference in their entirety.

### FIELD

The present disclosure relates to stearoyl-CoA Desaturase 1 (SCD1) inhibitors for treating medulloblastoma including high-risk medulloblastoma.

### BACKGROUND

Medulloblastoma (MB) is the most common malignant pediatric brain cancer and originates from the cerebellum¹. Genomic analysis has stratified MB into four distinct molecular subgroups; WNT, SHH, Group 3 (G3) and Group 4 (G4), which can be further divided into multiple subtypes. Treatment is limited to Standard of Care (SoC), consisting of surgical tumor resection followed by stringent chemotherapy and cranio-spinal radiation. Despite advancements in SoC and improved patient outcomes, 30% of MB patients succumb to the disease. Prognosis varies among subgroups, with MYC-driven G3-MB harboring the highest incidence of spinal metastasis and recurrence. Recurrent MB has a dismal 1-year survival rate of 40% and remains incurable^{2,3}. MB heterogeneity and recurrence is driven by a small subpopulation of cells termed brain tumor initiating cells (BTICs)^{4,5}. BTICs feature stem-like properties that fuel tumor formation and SoC resistance mechanisms such as DNA-repair and multidrug detoxification^{6,7}. MB survivors are plagued with treatment-induced life-long neurodevelopmental and neurocognitive impairments, and attest to the urgent need of improved therapeutic options^{3,8.}

The background herein is included solely to explain the context of the disclosure. This is not to be taken as an admission that any of the material referred to was published, known, or part of the common general knowledge as of the priority date.

### SUMMARY

The present application includes a method treating medulloblastoma (MB) comprising administering a therapeutically effective amount of a stearoyl-CoA Desaturase 1 (SCD1) inhibitor to a subject in need thereof.

The present application also includes a method of treating high-risk medulloblastoma comprising administering a therapeutically effective amount of a stearoyl-CoA Desaturase 1 (SCD1) inhibitor to a subject in need thereof.

In some embodiments, the high-risk medulloblastoma comprises a G3-MB, a G4-MB, G3 medulloblastoma that is MYC amplified (MYC-driven G3-MB), or a G4 medulloblastoma that is MYC amplified (MYC-driven G4-MB). In some embodiments, the high risk medulloblastoma comprises MYC-driven Group 3 MB (G3-MB) or MYC-driven Group 4 MB (G4-MB) in a subject in need thereof.

In some embodiments, the SCD1 inhibitor is any agent that inhibits expression of SCD1 gene or protein, that induces SCD1 protein degradation or that inhibits SCD1 protein activity.

In some embodiments, the SCD1 inhibitor is a small molecule inhibitor of SCD1 protein activity, or a pharmaceutically acceptable salt, solvate and/or prodrug thereof.

In some embodiments, treating high-risk medulloblastoma further comprises administering a therapeutically effective amount of an SCD1 inhibitor in combination with another known agent useful for treating (high-risk) medulloblastoma.

In some embodiments, treating (high-risk) medulloblastoma further comprises administering a therapeutically effective amount of an SCD1 inhibitor in combination with an agent useful for inhibiting de novo lipid synthesis,

In some embodiments, the another known agent useful for treating medulloblastoma is inhibitor of lipid metabolism, inhibitor of glycine metabolism, inhibitor of serine metabolism, or inhibitor of threonine metabolism.

In some embodiments, the another known agent useful for treating medulloblastoma is an inhibitor of phosphoglycerate dehydrogenase (PHGDH).

In some embodiment, treating high-risk medulloblastoma further comprises administering a therapeutically effective amount of an SCD1 inhibitor in combination with an agent useful for inhibiting pro-growth signaling cascades, including mitogen-induced kinases (MAPK, ERK1/2), JNK, Akt, β-catenin, GSK3b, or JNK.

The present application further comprises a pharmaceutical composition comprising an effective amount of a SCD1 inhibitor and a pharmaceutically acceptable carrier wherein the SCD1 inhibitor is present in amount effective to treat medulloblastoma.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should be given the broadest interpretation consistent with the description as a whole.

### DRAWINGS

Certain embodiments of the disclosure will now be described in greater detail with reference to the attached drawings in which:

FIG. 1 shows the G3 MB tumor cell lipidome displays a decreased abundance of saturated lipids when compared to NSC cells in exemplary embodiments of the disclosure. a Graph showing Principal Component Analysis (PCA) of untargeted lipidomics data. Row-normalized values of matrix without blank or NCC (after background subtraction). Graph shows cell line by dots and group by ellipse. b Heatmap of lipids with significantly different abundance (n=172/680 with adjusted p-value<0.05 (5% FDR) and log 2FC>|1|(FC>|2|)) from the following main lipid categories: fatty acyls, glycerolipids, glycerophospholipids, prenol lipids, sphingolipids and sterol lipids. c Graphs showing fatty acid (FA) composition of Triacylglycerols (TGs) (n=99): sum saturation level and carbon chain length. Composition plot of saturation level and carbon chain lengths of lipid main class Triacylglycerols (TG) at sum composition level of fatty acyl chains, proportional to each sample. Average abundance (Area Ratio Analyte/Internal Standard) of each sample triplicates is shown proportional to each sample. TG (n=99), Carbon chain length (n=38-66), Saturation level (double bonds, n=0-8). hNSCs (top three graphs), MBs (bottom three graphs).

FIG. 2 shows The De Novo Lipid Synthesis Pathway (DNL) is essential in G3 MB and pharmacological inhibition of SCD1 selectively targets G3 MB in exemplary embodiments of the disclosure. a Schematic representation of the De Novo Lipid synthesis (DNL) Pathway (made with BioRender®). b Visualization of gene essentiality of enzymes in the DNL pathway from genome-wide CRISPR-Cas9 KO Loss-of-function screen (TKOv3 library) in SU_MB002,⁴³ and mined CRISPR data set on NSCs⁴⁷ Bayes factor (BF) for SU_MB002, and average BF for NSCs (CB660 and U5), after quantile normalization c, d Transcriptomic expression of DNL enzymes in the HD-MB03 in vivo PDX standard of care (SoC) model, brain samples taken after engraftment and relapse. c Heatmap with hierarchical clustering (Z-score). d Boxplot with statistical comparison. Expression of SCD, ACLY, FASN and ACACA (TMM-normalized counts+log 2 transformation) followed a normal distribution (Shapiro-Wilk normality test). F-test to determine homogeneity of samples was performed; equality of variances was assumed for SCD, FASN and ACACA but not for ACLY). An independent t-test for equal and unequal variances was performed; accordingly, t-test p-values: 2.526e-05 for SCD, 0.08772 (ns) for ACLY, 0.007514 for FASN, 0.005108 for ACACA. e-h Dose-response curves on SU_MB002, HD-MB03 and NSC197 after 72 hrs treatment with e BMS, f TOFA, g C75 and h CAY10566. Cell viability is determined as residual PrestoBlue® reduction, normalized to vehicle treated cells (%). IC50 concentrations are determined by non-linear regression. ACLY, ATP citrate synthase; ACCα, Acetyl-Coa Carboxylase; FASN, Fatty Acid Synthase; SCD1, Stearoyl-Coa Desaturase 1; MUFA, mono-unsaturated fatty acid; PUFA, poly-unsaturated fatty acids. ACLY, ATP citrate synthase; ACCα, Acetyl-Coa Carboxylase; FASN, Fatty Acid Synthase; SCD1, Stearoyl-Coa Desaturase 1; MUFA, mono-unsaturated fatty acid; PUFA, poly-unsaturated fatty acids. See also FIG. 3.

FIG. 3 shows pharmacological Inhibition of SCD1 Impairs G3 MB Cell Viability and Self-Renewal In Vitro and is Rescued by Oleic Acid (OA) in exemplary embodiments of the disclosure. a Table indicating Bayes factor values of SU_MB002 and NSC CRISPR screens. b, c Treatment of HD-MB03 cells with the SCD1 inhibitor CAY10566. b Cell Growth assay. Normalized as % growth compared to Day 0 in AAVS1 and SCD1 KO SU_MB002 cells. Comparisons between DMSO and treatment conditions were made via two-way ANOVA Dunnett's multiple comparisons test, *p=0.0117 and ****p<0.0001, n=4. c Self-Renewal assay, sphere numbers quantified as % of DMSO control after 72 hrs incubation. Comparisons between DMSO and treatment condition were made via one-way ANOVA Dunnett's multiple comparison, n=4. c, d Cell viability assay on CAY10566 treated d SU_MB002 and e HD-MB03 cells, supplemented with 50 μM Oleic Acid (OA). Cell viability is determined as residual PrestoBlue®reduction, normalized to vehicle treated cells (%). IC50 concentrations are determined by non-linear regression. f Representative images of the SU_MB002 dose response curves of CAY10566 and OA, as seen in d.

FIG. 4 shows testing four TKOv3 CRISPR library SCD1 guides in exemplary embodiments of the disclosure. a Self-renewal assay. SCD1 KO (4 guides) sphere numbers quantified as % of AAVS1 control in HD-MB03 after 72 hrs incubation. Comparisons to AAVS1 were made via one-way ANOVA Dunnett's multiple comparisons test, ****p<0.0001, n=4. b Cell growth assay. SCD1 KO (4 guides) compared to AAVS1 KO (% growth compared to Day 0) in HD-MB03. Comparisons between DMSO and treatment conditions were made via two-way ANOVA Dunnett's multiple comparisons test, *p=0.0117 and ****p<0.0001, n=4. c Western Blot of the four TKOv3 SCD1 KO in HD-MB03.

FIG. 5 shows SCD1 KO Impairs G3 MB Self-Renewal and Viability in-vitro, and KO effects are rescued by Oleic Acid (OA) in exemplary embodiments of the disclosure. a Self-Renewal assay. SCD1 KO sphere numbers quantified as % of AAVS1 control in SU_MB002 after 72 hrs incubation. Comparison to AAVS1 was made via an unpaired t-test, ****P<0.0001, n=4 b Cell growth assay. SCD1 KO compared to AAVS1 KO (% growth compared to Day 0) in SU_MB002. Comparison between AAVS1 and SCD1 KO was made via two-way ANOVA Sidak multiple comparisons test, **p=0.0085, n=4. c Self-Renewal assay, SCD1 KO sphere numbers quantified as % of AAVS1 control in HD-MB03 after 72 hrs incubation. Comparison to AAVS1 was made via an unpaired t-test, ***P<0.0003, n=4 d Cell growth assay. SCD1 KO compared to AAVS1 KO (% growth compared to Day 0) in HD-MB03. Comparison between AAVS1 and SCD1 KO was made via two-way ANOVA Sidak multiple comparisons test, ***p=0.0001, n=4. e Western Blot indicating SCD1 protein expression in SU_MB002 and HD-MB03 AAVS1 control and SCD1 KO cells. Vinculin is used as loading control. f-i Self-renewal and Cell growth of HD-MB03 SCD1 KO cells (4 guides) with and without NCC media supplementation with Oleic Acid (OA). f Self-renewal assay. SCD1 KO (4 guides) sphere numbers quantified as % of AAVS1 control in HD-MB03 after 72 hrs incubation. g Self-renewal assay supplemented with Oleic Acid (OA). SCD1 KO (4 guides) sphere numbers quantified as % of AAVS1 control in HD-MB03 after 72 hrs. h Cell growth assay. SCD1 KO (4 guides) compared to AAVS1 KO (% growth compared to Day 0) in HD-MB03. i Cell growth assay supplemented with OA. SCD1 KO (4 guides) compared to AAVS1 KO (% growth compared to Day 0) in HD-MB03. See also FIG. 4.

FIG. 6 shows in vivo Pooled SCD1 KO survival and in vitro clonal SCD1 KO assay in exemplary embodiments of the disclosure. a, b Kaplan-Meier survival analysis of mice xenografted with (a) SU_MB002 and (b) HD-MB03 SCD1 and AAVS1 pooled KO tumors. Log-rank tests, **p=0.0043 & 0.0031, n=5-6 mice. c Representative H&E-stained sections of time-matched tissues of SCD1 KO and AAVS1 mouse brains (SU_MB002 and HD-MB03). d Tumor area (mm²) of time-matched brain tissue sections (SU_MB003 n=3-4, HD-MB03 n=2-3). An Unpaired 1-tailed t-test was performed (***p=0.0008 and * p=0.0366. (e-j) in vitro Oleic Acid (OA) rescue experiments for validation of SCD1 clonal KO cells. e Western blot comparing SCD1 expression in HD-MB03 AAVS1 and clonal SCD1 KO cells. Vinculin is used as loading control. f+i Self-renewal assay supplemented with OA. SCD1 single clone KO sphere numbers quantified as % of AAVS1 single clone control in f HD-MB03 and (I) SU_MB002 after 72 hrs. Comparisons between conditions with and without OA were made via one-way ANOVA Tukey's multiple comparisons test, **p=0.0013 and ****p<0.0001, n=4. (h) Western blot comparing SCD1 expression in SU_MB002 AAVS1 and clonal SCD1 KO cells. Vinculin is used as loading control, g, j Cell growth assay supplemented with Oleic Acid (OA). SCD1 single KO cells compared to AAVS1 single KO cells (% growth compared to Day 0) in g HD-MB03 and j SU_MB002. Comparisons between conditions with and without OA after 168 hrs were made via one-way ANOVA Tukey's multiple comparisons ****p<0.0001, n=4 (k) Representative images of HD-MB03 and SU_MB002 clonal SCD1 KO cells. Note that HD-MB03 cells consist mostly of spheres rather than adherent Images taken by EVOS, scale is 1000 μM.

FIG. 7 shows clonal KO and Pharmacological inhibition of SCD1 effectively improves survival in G3 MB in vivo in exemplary embodiments of the disclosure. a Schematic indicating the derivation of SCD1 single clone KO cells by FACS sorting and subsequent intracranial injection. b,c Kaplan-Meier survival analysis of mice intracranially engrafted with SCD1 and AAVS1 single clone KO b HD-MB03 and c SU_MB002 tumors. (Log-rank tests, ***p=0.0002 and **p=0.0012, n=6-7 mice. d Schematic indicating the murine intracranial tumor model, with CAY10566 intraperitoneal (IP) treatment. e CAY10566 IP treatment (20 mg/kg) regimen and IVIS imaging schedule for the intracranial murine model. f Kaplan-Meier survival analysis Kaplan-Meier survival analysis of intracranial SU_MB002 FFLUC tumors, treated with CAY10566, Log-rank tests, ***p=0.0010 n=5-6 mice. g Optimized CAY10566 IP treatment (20 mg/kg) regimen and IVIS imaging schedule for the intracranial model. 6, h IVIS imaging results of optimized CAY10566 dosing regimen, plotted as Fold change (in total Flux) based on initial IVIS imaging (n=3). i Luciferase bioluminescence images by IVIS of optimized CAY10566 treatment regimen at the indicated days. Three days post injections mice were dosed via IP injection with 20 mg/kg CAY10566. Illustrations made with BioRender®. See also FIGS. 6 and 8.

FIG. 8 shows CAY10566 treatment in in vivo intracranial and Flank murine models in exemplary embodiments of the disclosure. a Schematic indicating the murine intracranial tumor model, with CAY10566 intraperitoneal (IP) treatment. All intracranial experimental results are outlined in grey. b Kaplan-Meier survival analysis Kaplan-Meier survival analysis of intracranial HD-MB03 FFLUC tumors, treated with CAY10566, Log-rank tests *p=0.0102, n=5-6 mice. c, d IVIS imaging results of intracranial CAY10566 dosing regimen, plotted as Fold change (in total Flux) based on initial IVIS imaging (n=5-6) in mice bearing c SU_MB003 FFLUC tumors and d HD-MB03 FFLUC tumors. e Schematic indicating the murine flank tumor model, with CAY10566 intraperitoneal (IP) treatment. All flank experimental results are outlined in blue. f, g IVIS imaging results of flank CAY10566 dosing regimen, plotted as Fold change (in total Flux) based on initial IVIS imaging (n=5-6) in mice bearing f SU_MB003 FFLUC and g HD-MB03 FFLUC tumors.

FIG. 9 shows differentially expressed genes and enriched pathways after CAY10566 treatment in G3 MB in exemplary embodiments of the disclosure. DEGs obtained after performing differential expression analysis using DESeq2 on SU_MB002 cells by treatment (SCD1 inhibitor CAY10566 4 nM vs vehicle DMSO as control). a Volcano plot using log 2FC>|1| adjusted value<0.05 b Heatmap of the DEGs: 138 up-regulated and 28 down-regulated genes. Cut-off after DE analysis: FDR 5% (p-adjusted<0.05), Log2FC>|1|. Gene set enrichment analysis was performed with the list of DEGs (MB002 treatment vs vehicle) for all DEGs (n=162) for the c KEGG, and the d GSEA MSigDB Hallmark of cancer databases. Significantly enriched pathways are shown in a dot plot with the gene count and adjusted value. e Phospho-Kinase Array Dot plots of phosphoproteins of SU_MB002 SCD1 KO vs AAVS1 KO cell lysates f Bar graph of Phospho-Kinase Array Dot plot mean pixel densities. Two-way ANOVA multiple comparisons, n=2 independent replicates, adjusted **** p=<0.0001. g Bar graph showing Annexin V positivity versus viability in SU_MB002 cells treated with CAY10566 or DMSO for 72 hrs. h Bar graph showing cell-cycle phase (DNA content, PI staining) after 72 hrs of treatment with CAY10566 or DMSO control. Percentages derived from FACS analysis. See also FIG. 10.

FIG. 10 shows differentially expressed genes and enriched pathways after CAY10566 treatment in G3 MB in exemplary embodiments of the disclosure. a Enriched pathways and gene symbols in RNA-seq analysis of SU_MB002 CAY10566 (IC₅₀ vs DMSO), using KEGG and GSEA MSigDB Hallmark of cancer databases. Downregulated genes are highlighted in blue. b Phospho-Kinase Array Dot plots of phosphoproteins of SU_MB002 CAY10566 treated vs DMSO cell lysates c Bar graph of Phospho-Kinase Array Dot plot mean pixel densities. Two-way ANOVA multiple comparisons, n=2 independent replicates, adjusted **** p=<0.0001.

FIG. 11 shows SCD gene expression is a prognostic marker in Group3 and Group 4 MB molecular subgroups in exemplary embodiments of the disclosure. a Principal component analysis of normalised microarray expression data from 763 MB patients (70 WNT, 223 SHH, 144 Group-3 and 326 Group-4) from the Cavalli dataset (GSE85217)⁶⁵. b Boxplots of SCD expression by molecular classification in subtypes (49 WNT-a, 21 WNT-b; 65 SHH-a, 35 SHH-b, 47 SHH-g, 76 SHH-d; 67 G3-a, 37 G3-b, 40 G3-g; 98 G4-a, 109 G4-b, 119 G4-g). Shapiro-Wilk normality test p-value=0.1567. One-way ANOVA for Molecular subtype (<2e-16***), Tukey HSD post-hoc test in Supplementary Table X. c-g Kaplan-Meier curves showing overall survival (OS) probability based on patients with very high (>Q3) or very low (<Q1) SCD expression using the quartiles from each cohort as threshold. Survival analysis was performed for c G3 n=58, d G4 n=134, e G3+G4 n=193, f WNT n=32 and g SHH n=88. Analysis was performed after removing samples with NA info in Event (0,1) and/or survival(time). The Cox regression model for survival analysis was used, p-values (p), Hazard-Ratio (HZ) and 95% confidence interval (CI) for the statistically significant comparison are shown. h Correlation of SCD and MYC gene expression across MB patient samples for both MB molecular subgroups G3 and G4, and pooled G3+G4. See also FIG. 12.

FIG. 12 shows SCD expression correlates with MYC expression in MB-G3 in exemplary embodiments of the disclosure. Correlation of SCD and MYC gene expression a across all 763 MB samples from the Cavalli dataset, and samples subsets by b Molecular subgroup and c molecular subtype. The Pearson correlation method was used.

FIG. 13 shows medulloblastoma G3 and G4 patients with distinct SCD expression may use different metabolic programs in exemplary embodiments of the disclosure. a Volcano plot showing the results from the differential expression analysis comparing G3+G4 MB patients with very high to very low SCD expression (the later used as control). Threshold used: adjusted value<0.01 and log 2FC>|1|. (b) Heatmap with hierarchical clustering of the 120 DEGs with clinical and molecular annotations. c, d Significantly enriched pathways identified after performing gene set enrichment analysis with c 47 up-regulated and (d) 73 down-regulated DEGs, using the “MiSigDB Hallmark 2020”, “KEGG” and “TF perturbations followed by expression” databases from Enrichr®⁸⁶⁻⁸⁸.

FIG. 14 shows phosphoglycerate dehydrogenase (PHGDH) is a target in MYC-amplified G3-MB and synergizes with CAY10566. A Dose-response curve of NCT-503 on SU_MB002 cells. B Dose-response curve of NCT-503 on HD-MB03. Three-fold serial dilutions were performed to yield final nine concentrations starting from 25 μM. C Synergy assay performed twice on SU_MB002 with CAY10566 (SCD1 inhibitor) versus NCT-503 (PHGDH inhibitor). Concentrations used were based on drug IC50 values. Bliss synergy scores higher than 10 are synergistic.

### DETAILED DESCRIPTION

### I. Definitions

Unless otherwise indicated, the definitions and embodiments described in this and other sections are intended to be applicable to all embodiments and aspects of the present disclosure herein described for which they are suitable as would be understood by a person skilled in the art. It is also to be understood that the terminology used herein is for the purpose of describing particular aspects only and is not intended to be limiting.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. In addition, all ranges given herein include the end of the ranges and also any intermediate range points, whether explicitly stated or not.

As used in this disclosure, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

The term “subject” as used herein includes all members of the animal kingdom including mammals. Thus, the methods and uses of the present application are applicable to both human therapy and veterinary applications.

The term “pharmaceutically acceptable” means compatible with the treatment of subjects.

The term “pharmaceutically acceptable carrier” means a non-toxic solvent, dispersant, excipient, adjuvant or other material which is mixed with an active ingredient to permit the formation of a pharmaceutical composition, i.e., a dosage form capable of administration to a subject.

The term “pharmaceutically acceptable salt” means either an acid addition salt or a base addition salt of a compound which is suitable for, or compatible with the treatment of subjects.

An acid addition salt suitable for, or compatible with, the treatment of subjects is any non-toxic organic or inorganic acid addition salt of any basic compound.

A base addition salt suitable for, or compatible with, the treatment of subjects is any non-toxic organic or inorganic base addition salt of any acidic compound.

The term “prodrug” as used herein means a compound, or salt and/or solvate of a compound, that, after administration, is converted into an active drug.

The term “solvate” as used herein means a compound, or a salt or prodrug of a compound, wherein molecules of a suitable solvent are incorporated in the crystal lattice.

The term “treating” or “treatment” as used herein and as is well understood in the art, means an approach for obtaining beneficial or desired results, including clinical results. Beneficial or desired clinical results can include, but are not limited to alleviation or amelioration of one or more symptoms or conditions, diminishment of extent of a disease, disorder or condition, stabilized (i.e. not worsening) state of a disease, disorder or condition, preventing spread of a disease, disorder or condition, delay or slowing of a disease, disorder or condition progression, amelioration or palliation of a disease, disorder or condition state, diminishment of the reoccurrence of a disease, disorder or condition, and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with cancer is treated to prevent progression, or alternatively a subject who has had cancer is treated to prevent recurrence.

“Palliating” a disease, disorder or condition means that the extent and/or undesirable clinical manifestations of the disease, disorder or condition are lessened and/or time course of the progression is slowed or lengthened, as compared to not treating the disease, disorder or condition.

The term “preventing”, “prevention” or “prophylaxis”, or synonym thereto, as used herein refers to a reduction in the risk or probability of a subject becoming afflicted with a disease, disorder or condition or manifesting a symptom associated with a disease, disorder or condition, and includes blocking the onset of the disease, disorder or condition, and/or to a reduction in the risk or probability of reoccurrence of the disease, disorder or condition in a subject that has had the disease, disorder or condition.

As used herein, the term “effective amount” or “therapeutically effective amount” means an amount of a SCD1 inhibitor that is effective, at dosages and for periods of time necessary to achieve the desired result.

By “inhibiting” it is meant any detectable inhibition, slowing and/or disruption in the presence of the SCD1 inhibitor compared to otherwise the same conditions, except for in the absence in the SCD1 inhibitor.

The term “SCD1” as used herein refers to STEAROYL-COA DESATURASE-1.

The term “administered” as used herein means administration of a therapeutically effective amount of a SCD1 inhibitor, or a composition thereof to a cell or a subject.

The term “CAY10566” as used herein refers to a compound having the chemical name: 3-[4-(2-chloro-5-fluorophenoxy)-1-piperidinyl]-6-(5-methyl-1,3,4-oxadiazol-2-yl)-pyridazine and having the chemical formula:

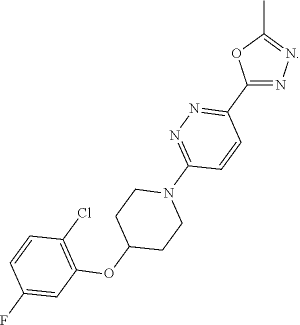 [SMILES: Cc1nnc(-c2ccc(N3CCC(Oc4cc(F)ccc4Cl)CC3)nn2)o1]

The term “BMS303141” as used herein refers to a compound having the chemical name: 3,5-Dichloro-2-hydroxy-N-(4-methoxy[1,1′-biphenyl]-3-yl)-benzenesulfonamide and having the chemical formula:

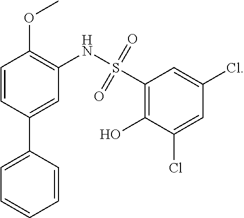 [SMILES: COc1ccc(-c2ccccc2)cc1NS(=O)(=O)c1cc(Cl)cc(Cl)c1O]

The term “TOFA” as used herein refers to a compound having the chemical name: 5-(tetradecyloxy)-2-furoic acid and having the chemical formula:

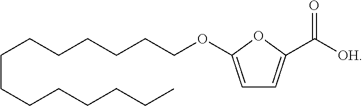 [SMILES: CCCCCCCCCCCCCCOc1ccc(C(=O)O)o1]

The term “C75” as used herein refers to a compound having the chemical name: 4-Methylene-2-octyl-5-oxotetrahydrofuran-3-carboxylic acid and having the chemical formula:

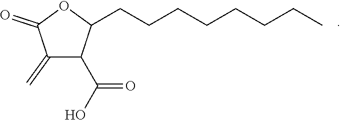 [SMILES: C=C1C(=O)OC(CCCCCCCC)C1C(=O)O]

The term “NCT-503” as used herein refers to a compound having the chemical name: N-(4,6-Dimethylpyridin-2-yl)-4-(4-(trifluoromethyl)benzyl)piperazine-1-carbothioamide and having the chemical formula:

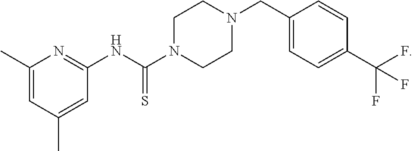 [SMILES: Cc1cc(C)nc(NC(=S)N2CCN(Cc3ccc(C(F)(F)F)cc3)CC2)c1]

It will be understood that any component defined herein as being included may be explicitly excluded by way of proviso or negative limitation, such as any specific compounds or method steps, whether implicitly or explicitly defined herein.

### II. Methods and Uses of the Application

To gain molecular insights in MB disease progression and relapse, a recently developed murine patient-derived orthotopic xenograft (PDOX) model that is adapted to SoC was used. Transcriptomic analysis at different disease stages (engraftment, post-chemotherapy, post-chemoradiotherapy, and recurrence) revealed differences in metabolic and lipogenic pathways⁹. This apparent metabolic reprogramming enables tumor cells to gain independence from limited environmental and nutritional conditions by de novo production of metabolites^{10,11}. Within this context, lipid metabolism plays a central role in sustaining the tumor cell's high Fatty Acid (FA) demand for energy production, structural components, and resistance to hypoxia¹².

The De Novo Lipid synthesis (DNL) pathway is a crucial component in cancer cell survival. The DNL pathway involves a sequence of fatty acid biosynthetic enzymes: ATP-citrate lyase (ACLY), Acetyl-CoA carboxylase (ACC), Fatty Acid Synthase (FASN) and stearoyl-CoA Desaturase 1 (SCD1)¹³⁻¹⁶. Together, these enzymes produce and elongate fatty acids (FAs) that are derived from the non-lipid precursor acetyl coenzyme-A (Acetyl-CoA)^{17,18}.

SCD1 is the most prominent of Δ9-Desaturases and is located at the endoplasmic reticulum^{17,18}. Here it converts saturated FAs, such as palmitoyl-CoA and stearoyl-CoA, into Δ9-monounsaturated fatty acids (MUFAs), with Oleic Acid (OA) as one of its main products¹⁹⁻²¹. SCD1 has been described in various cancers, including hepatocellular carcinoma, renal cell carcinoma, breast cancer, bladder cancer, melanoma, glioblastoma (GBM), pancreatic, lung cancer, colon cancer, and gastric cancer²²⁻³¹. Increased SCD1 expression correlates with highly aggressive phenotypes and poor prognosis in liver, thyroid, prostate, pancreatic, kidney, skin, and breast cancer³²⁻³⁶. These pro-tumorigenic features can be explained by the involvement of SCD1 in cancer cell proliferation, migration, metastasis, and tumor growth^{18,37} Importantly, the DNL pathway protects cancer cells from free radicals and chemotherapeutic agents by promoting membrane lipid saturation³⁸. Through this mechanism, therapeutic targeting of SCD1 acts as a sensitizer to SoC¹⁸.

FA exist in two classes: saturated (SFAs) and unsaturated (UFAs), displaying a vast variety of saturation levels and chain lengths^{17,18}. FAs are central structural components of all cellular lipid bilayers and membranes. They are incorporated into larger molecules such as sterols, glycerophospholipids, sphingolipids and triglycerides, which maintain membrane fluidity. Additionally, they function as an energy source to fuel p-oxidation and are involved in numerous signaling pathways, such as endocytosis, exocytosis and cell survival³⁹⁻⁴¹. Cancer cells maintain a homeostatic balance between SFAs and UFAs that is crucial to their survival. This balance prevents lipotoxicity and subsequent ferroptosis, indicating that targeting the DNL pathway in cancer cells may be a viable therapeutic option⁴²

Here untargeted Liquid-Chromatography-Mass Spectrometry (LC-MS) was employed to explore the G3-MB lipidome. Lipidomic profiles of three patient derived G3-MB cell lines (SU_MB002, HD-MB03 and MED411 FHTC) and three normal human Neural Stem Cell (hNSC) cell lines (hNSC194, hNSC197 and hNSC201) were compared. It was determined that G3-MB presents a differential abundance of distinct lipid species with varying degrees of desaturation. These differences indicated a potential dysregulation of the DNL pathway. DNL enzyme essentiality in MB was confirmed by probing the previously conducted genome-wide CRISPR-Cas9 directed loss of function screen⁴³. Moreover, a SoC-adapted patient-derived xenograft (PDX) murine model to recapitulate disease progression through treatment and relapse was recently developed⁹. RNA-seq analysis comparing tumor cells harvested at engraftment and relapse revealed significantly upregulated expression of the DNL enzymes at relapse. Surprisingly, drug screening revealed SCD1 as a potent therapeutic target for G3-MB. Furthermore, small molecule treatment targeting SCD1 was shown to demonstrate efficacy against G3-MB PDX models in vivo. Lastly, high SCD expression was identified to be a prognostic marker and revealed SCD regulated metabolic pathways by utilizing a patient tumor dataset

Therefore, the Applicants have surprisingly found a differential abundance of lipid species and lipid saturation levels between NSC and G3-MB cells leading to the identification of the DNL pathway as a therapeutic vulnerability in MB (e.g. G3-MB). Thus, discovering a therapeutic opportunity in medulloblastoma. Further, the Applicants have identified SCD1 to be the most essential gene of the DNL pathway in MBs but not in NSCs, and further found SCD expression to be a prognostic marker for poor survival in G3-MB and G4-MB. Further, the Applicants have shown by using SCD1 inhibitors such as CAY10566 that SCD1 inhibition selectively targets MB BTICs over normal NSC demonstrating SCD1 to be a targetable vulnerability only in G3-MB, while sparing healthy NSC populations. These results confirm SCD1 as a potent therapeutic target for MB, for example, G3 and G4 MB. Further, cholesterol homeostasis; myogenesis; glycine, serine, and threonine metabolism pathways were identified as potent targets for combinatorial treatment with SCD1 inhibitors.

The present application includes a method of treating medulloblastoma (MB) comprising administering a therapeutically effective amount of a stearoyl-CoA Desaturase 1 (SCD1) inhibitor to a subject in need thereof. The present application also includes a use of a SCD1 inhibitor for treating medulloblastoma as well as a use of a SCD1 inhibitor for the preparation of a medicament for treating medulloblastoma. The application further includes SCD1 inhibitor for use for treating medulloblastoma.

In some embodiments, the medulloblastoma is high-risk medulloblastoma. Therefore, the present application includes a method of treating high-risk medulloblastoma (MB) comprising administering a therapeutically effective amount of a stearoyl-CoA Desaturase 1 (SCD1) inhibitor to a subject in need thereof. The present application also includes a use of a SCD1 inhibitor for treating high-risk medulloblastoma as well as a use of a SCD1 inhibitor for the preparation of a medicament for treating high-risk medulloblastoma. The application further includes SCD1 inhibitor for use for treating high-risk medulloblastoma

In some embodiments, the high-risk medulloblastoma is a medulloblastoma in a subject that is less than three years old, a medulloblastoma that has metastasized (e.g. M+ disease), a residual medulloblastoma from incomplete resection with a residual amount of tumour>1.5 cm², large cell/anaplastic (LCA) medulloblastoma, a recurrent medulloblastoma, medulloblastoma with amplification of MYC or MYCN, a Group 3 (G3) medulloblastoma, or a Group 4 (G4) medulloblastoma or a combination thereof.

In some embodiments, the high-risk medulloblastoma is a recurrent medulloblastoma, medulloblastoma with amplification of MYC, a Group 3 (G3) medulloblastoma, or a Group 4 (G4) medulloblastoma or a combination thereof. In some embodiments, the high-risk medulloblastoma is a G3 medulloblastoma, a G4 medulloblastoma, G3 medulloblastoma that is MYC amplified (MYC-driven G3-MB), or a G4 medulloblastoma that is MYC amplified (MYC-driven G4-MB). In some embodiments, the high risk medulloblastoma is recurrent medulloblastoma.

In some embodiments, the high risk medulloblastoma comprises MYC-driven Group 3 MB (G3-MB) or MYC-driven Group 4 MB (G4-MB) in a subject in need thereof.

In some embodiments, treating high-risk medulloblastoma comprises inhibiting de novo lipid synthesis (optionally, the de novo lipid synthesis (DNL) pathway). In some embodiments, treating high-risk medulloblastoma comprises inhibiting SCD1.

In an embodiment, the subject is a mammal. In another embodiment, the subject is human.

In some embodiments, the subject in need thereof is a subject having a (high-risk) medulloblastoma or a subject that has had a (high-risk) medulloblastoma. In some embodiments, the subject in need thereof is a subject having a (high-risk) medulloblastoma. In some embodiments, the subject in need thereof is a subject having the (high-risk) medulloblastoma that has metastasized. In some embodiments, the subject in need thereof is a subject having the (high-risk) medulloblastoma and the cancer is in remission. In some embodiments, the subject in need thereof is a subject having a residual medulloblastoma from incomplete resection with a residual amount of tumour>1.5 cm² In some embodiments, the subject in need thereof is a subject that is cancer-free after having had the (high-risk) medulloblastoma. In some embodiments, the subject in need thereof is a subject having a (high-risk) medulloblastoma and the cancer is refractory to a chemotherapy or radiotherapy. In some embodiments, the subject in need thereof is a subject having a medulloblastoma and is less than 3 years old. In some embodiments, the subject in need thereof is a subject having a G3-MB, G4-MB, MYC-driven G3-MB or MYC-driven G4-MB.

In some embodiments, the SCD1 inhibitor is administered or used as soon as possible after MB diagnosis. In some embodiments, the SCD1 inhibitor is administered or used as soon as possible after a subject that has had MB is in remission or is cancer-free.

In some embodiments, the SCD1 inhibitor is any agent that inhibits expression of SCD1 gene or protein, that induces SCD1 protein degradation or that inhibits SCD1 protein activity.

In some embodiments, the SCD1 inhibitor is any agent that inhibits expression of SCD1 gene or protein. In some embodiments, inhibiting expression of SCD1 gene or protein is by changing the content of DNA or degrading mRNA. In some embodiments, inhibiting expression of SCD1 gene or protein is by SCD1 gene knockdown, SCD1 gene knockout or by IMPDP gene editing.

In some embodiments, the agent that inhibits expression of SCD1 gene or protein is a nucleic acid. In some embodiments, the human SCD1 is encoded by gene Ensembl:ENSG00000099194 MIM:604031; AllianceGenome:HGNC:10571,Homo sapiens) (Dyer S C et al. “Ensembl 2025” NucleicAcids Res. 2025, 53(D1):D948-D957). In some embodiments, the nucleic acid is an antisense oligonucleotide. In some embodiments, the antisense oligonucleotide is complementary to an SCD1 DNA or RNA sequence or a variant or fragment thereof.

In some embodiments, the agent that inhibits expression of SCD1 gene or protein is a nucleic acid selected from small interfering RNA (siRNA), dicer substrate DNA, hairpin RNA, microRNA (miRNA), RNAi and splice-regulating oligonucleotides. In some embodiments, the agent that inhibits expression of SCD1 gene or protein is selected from small interfering RNA (siRNA) and microRNA (miRNA).

In some embodiments, the agent that inhibits expression of SCD1 gene or protein is a gene editing system. In some embodiments, the gene editing system is a clustered regularly interspaced short palindromic repeat (CRISPR)-Cas system (CRISPR system); zinc finger nuclease (ZFN) system, or transcription activator-like effector-based nuclease (TALEN) system.

In some embodiments, the agent that inhibits expression of SCD1 gene or protein is a CRISPR system.

In some embodiments, the agent that inhibits expression of IMPDH protein or inhibits SCD1 protein activity is an antibody that specifically binds SCD1 or an antigen binding fragment thereof.

As used herein, the term “antibody” refers to immunoglobulin molecules and immunologically active portions of immunoglobulin (Ig) molecules, i.e., molecules that contain an antigen binding site that specifically binds an antigen (for example, SCD1). Such antibodies include, but are not limited to, polyclonal, monoclonal, chimeric, single chain, Fab, Fab′ and F(ab′)2 fragments, an Fab expression library, single-chain antibody molecules (e.g., scFv), bispecific antibodies and antibody-drug conjugates.

As used herein, the expression “specifically binds” means that the antibody reacts with one or more antigenic determinants of the desired antigen and does not bind other polypeptides or binds other polypeptides at much lower affinity (Kd>10⁻⁶).

In some embodiments, the SCD1 inhibitor is any agent that induces SCD1 protein degradation.

In some embodiments, the agent that induces SCD1 protein degradation (optionally SCD1 inhibitor) is a targeted SCD1 protein degrader. In some embodiments, the targeted SCD1 protein degrader is selected from an SCD1 targeting proteolysis targeting chimera (PROTAC), an SCD1 targeting molecular glue degrader, a selective estrogen receptor degrader (SERD), an SCD1 targeting monoclonal antibody or an SCD1 targeting antibody-drug conjugate.

In some embodiments, the targeted protein degrader is a small molecule targeted protein degrader selected from an SCD1 targeting proteolysis targeting chimera (PROTAC), an SCD1 targeting molecular glue degrader and a selective estrogen receptor degrader (SERD). In some embodiments, the targeted protein degrader is selected from an SCD1 targeting proteolysis targeting chimera (PROTAC), and an SCD1 targeting molecular glue degraders. In some embodiments, the targeted protein degrader is an SCD1 targeting monoclonal antibody or an SCD1 targeting antibody-drug conjugate. In some embodiments, the antibody-drug conjugate is antibody-PROTAC conjugate.

In some embodiments, PROTACs are bifunctional molecules that comprise a ligase binding group and a target protein binding group (e.g. an SCD1 binding group) which are joined together by a linker. By binding with high affinity to a target protein in the cell while binding with high affinity to the ligase, PROTACs function to recruit proteins (e.g., enzymes such as SCD1) to a ligase which are then degraded and/or otherwise inhibited by the bifunctional compounds. In exemplary embodiments, the ubiquitination ligase binding group is a Von Hippel-Lindau E3 ubiquitin ligase (VHL) binding group, a cereblon E3 ubiquitin ligase binding group, or mouse double minute 2 homolog (MDM2 or HDM2) E3 ubiquitin ligase binding group, or IAP E3 ubiquitin ligase binding group. Many examples of PROTACs are now known in the art for targets including kinases, hormone receptors, and proteases (see for example, Tsai, J., et al. Nat Rev Mol Cell Biol (2024), Lai et al., Nature Reviews Drug Discovery volume 16, pages 101-114 (2017).

In some embodiments, molecular glue degraders are monovalent bifunctional molecules comprising a ligase binding group and a target protein binding group but which do not comprise a linker. In some embodiments, molecular glue degraders bind the target protein and ligase through cooperative binding and reshape protein surface to enhance the affinity of the target protein and ligase for each other and/or promote novel protein-protein interactions. In some embodiments, the molecular glue degraders are naturally occurring protein degrader such Zinc²⁺ ions, viral peptides, auxins, RNA or hormones. In some embodiments, the molecular glue degraders are selected from thalidomide, lenolidamide, pomalidomine, and further thalidomide-based analogues such as CC-122, CC-220, CC-90009, CC-92480, ZXH-1-161, and SJ6986, dCeMM1-4, NRX-252114 and NRX-252262 and CR8 (see for example, Tsai, J., et al. Nat Rev Mol Cell Biol (2024), Sasso J, et al., Biochemistry. 2023 Feb. 7; 62(3): 601-623).

In some embodiments, selective estrogen receptor degraders (SERDs) bind to the estrogen receptor (ER) and induce a conformational change that results in the degradation and/or downregulation of the receptor. In some embodiments, the degradation of the ER prevents ER-mediated signaling and inhibits the growth and survival of ER-expressing cancer cells (see for example, Tsai, J., et al. Nat Rev Mol Cell Biol (2024).

In some embodiments, the SCD1 inhibitor is any agent that inhibits SCD1 protein activity. In some embodiments, the agent that inhibits SCD1 protein activity is a small molecule inhibitor of SCD1 protein activity or a pharmaceutically acceptable salt, solvate and/or prodrug thereof.

In some embodiments, the small molecule inhibitor of SCD1 protein activity (optionally, the SCD1 inhibitor) or a pharmaceutically acceptable salt, solvate and/or prodrug thereof is any SCD1 inhibitor known in the art. For example, in some embodiments, the SCD1 inhibitor is an inhibitor disclosed in Tracz-Gaszewska Z, et al., Cancers (Basel). 2019 Jul. 5; 11(7):948 and Tardiff D F et al., Mol Neurobiol. 2022 Apr; 59(4):2171-2189. In some embodiments, the small molecule inhibitor of SCD1 protein activity (optionally, the SCD1 inhibitor) or a pharmaceutically acceptable salt, solvate and/or prodrug thereof is selected from, but is not limited to, A939572, MF-438, CVT-11127, CVT-12012, CAY10566, T-3764518, BZ36, SSI-4, SW208108, SW203668 and YTX-7739. In some embodiments, the small molecule inhibitor of SCD1 protein activity (optionally, the SCD1 inhibitor) is CAY10566 or YTX-773970. In some embodiments, the small molecule inhibitor of SCD1 protein activity (optionally, the SCD1 inhibitor) is CAY10566.

In some embodiments, the present application also includes a method of treating medulloblastoma (optionally, high-risk medulloblastoma) comprising administering a therapeutically effective amount of a SC1 inhibitor in combination with another known agent useful for treating medulloblastoma (optionally, high-risk medulloblastoma) in a subject in need thereof.

The present application also includes a use of a SCD1 inhibitor in combination with another known agent useful for treating medulloblastoma (optionally, high-risk medulloblastoma) in a subject in need thereof, as well as a use of a SCD1 inhibitor in combination with another known agent useful for treating medulloblastoma (optionally, high-risk medulloblastoma) for the preparation of a medicament for treating medulloblastoma (optionally, high-risk medulloblastoma) in a subject in need thereof. The application further includes a SCD1 inhibitor in combination with another known agent useful for treating medulloblastoma (optionally, high-risk medulloblastoma) for use for treating high-risk medulloblastoma in a subject in need thereof.

In some embodiments, treating (optionally, high-risk medulloblastoma) further comprises administering a therapeutically effective amount of an SCD1 inhibitor in combination with another known agent useful for treating a high-risk medulloblastoma. In some embodiments, treating high-risk medulloblastoma further comprises administering a therapeutically effective amount of an SCD1 inhibitor in combination with another known agent useful for treating a high-risk medulloblastoma.

In some embodiments, the another known agent useful for treating medulloblastoma (optionally, high-risk medulloblastoma) is an agent useful for inhibiting de novo lipid synthesis. Therefore, in some embodiments, treating high-risk medulloblastoma further comprises administering a therapeutically effective amount of an SCD1 inhibitor in combination with another known agent useful for inhibiting de novo lipid synthesis.

In some embodiments, the another known agent useful for treating medulloblastoma (optionally, high-risk medulloblastoma) are inhibitors of lipid metabolism.

In some embodiments, the another known agent useful for treating medulloblastoma (optionally, high-risk medulloblastoma) are inhibitors of amino acid metabolism. In some embodiments, the another known agent useful for treating high-risk medulloblastoma are inhibitors of glycine metabolism, serine metabolism, or threonine metabolism.

In some embodiments, the another known agent useful for treating medulloblastoma (optionally, high-risk medulloblastoma) are inhibitors of lipid metabolism. In some embodiments, the inhibitor of lipid metabolism is a statin.

In some embodiments, the another known agent useful for treating medulloblastoma (optionally, high-risk medulloblastoma) are inhibitors of serine metabolism. In some embodiments, the another known agent useful for treating high-risk medulloblastoma are inhibitors of phosphoglycerate dehydrogenase (PHGDH) such as NCT-503.

In some embodiments, the another known agent useful for medulloblastoma (optionally, high-risk medulloblastoma) is an agent useful for inhibiting pro-growth signaling cascades, including mitogen-induced kinases (MAPK, ERK1/2), JNK, and Akt, or β-catenin, GSK3b, JNK. In some embodiments, treating high-risk medulloblastoma further comprises administering a therapeutically effective amount of an SCD1 inhibitor in combination with another known agent useful for inhibiting pro-growth signaling cascades, including mitogen-induced kinases (MAPK, ERK1/2), JNK, and Akt, or β-catenin, GSK3b, JNK.

In some embodiments, the another known agent useful for treating a high-risk medulloblastoma comprises surgical resection, chemotherapy or cranio-spinal irradiation.

In some embodiments, the another known agent useful for treating medulloblastoma (optionally, high-risk medulloblastoma) is a cancer treatment such as radiotherapy, chemotherapy, targeted therapies such as antibody therapies (including anti-PD1 and/or anti-PD-L1 antibodies) and small molecule therapies such as tyrosine-kinase inhibitors therapies, glutaminase inhibitors (e.g., glutaminase-1 (GLS1) inhibitors), and asparagine synthetase (ASNS) inhibitors, immunotherapy, hormonal therapy and anti-angiogenic therapies, and combinations thereof.

Treatment methods comprise administering to a subject a therapeutically effective amount of a SCD1 inhibitor optionally consist of a single administration, or alternatively comprise a series of administrations, and optionally comprise concurrent administration or use of one or more other therapeutic agents. For example, in some embodiments, a SCD1 inhibitor may be administered at least once a week. In some embodiments, the SCD1 inhibitor may be administered to the subject from about one time per two or three weeks, or about one time per week to about once daily for a given treatment. In another embodiment, the compounds are administered 2, 3, 4, 5 or 6 times daily. The length of the treatment period depends on a variety of factors, such as the severity of the disease, disorder or condition, the age of the subject, the concentration and/or the activity of the SCD1 inhibitor, and/or a combination thereof. It will also be appreciated that the effective dosage of the SCD1 inhibitor used for the treatment may increase or decrease over the course of a particular treatment regime. Changes in dosage may result and become apparent by standard diagnostic assays known in the art. In some instances, chronic administration may be required. For example, the SCD1 inhibitor is administered to the subject in an amount and for duration sufficient to treat the subject. In some embodiments treatment comprise prophylactic treatment. For example, a subject with early cancer can be treated to prevent progression, or alternatively a subject in remission can be treated with a compound or composition of the application to prevent recurrence.

The dosage of SCD1 inhibitor varies depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the SCD1 inhibitor in the subject to be treated. One of skill in the art can determine the appropriate dosage based on the above factors. A SCD1 inhibitor may be administered initially in a suitable dosage that may be adjusted as required, depending on the clinical response. A SCD1 inhibitor may be administered in a single daily, weekly or monthly dose or the total daily dose may be divided into two, three or four daily doses.

In an embodiment, effective amounts vary according to factors such as the disease state, age, sex and/or weight of the subject. In a further embodiment, the amount of a given compound or compounds that will correspond to an effective amount will vary depending upon factors, such as the given drug(s) or compound(s), the pharmaceutical formulation, the route of administration, the type of condition, disease or disorder, the identity of the subject being treated, and the like, but can nevertheless be routinely determined by one skilled in the art.

In some embodiments, the pharmaceutically acceptable salt is an acid addition salt or a base addition salt. The selection of a suitable salt may be made by a person skilled in the art (see, for example, S. M. Berge, et al., “Pharmaceutical Salts,” J. Pharm. Sci. 1977, 66, 1-19).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of an SCD1 inhibitor for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. The selection of the appropriate salt may be useful, for example, so that an ester functionality, if any, elsewhere in a compound is not hydrolyzed. The selection criteria for the appropriate salt will be known to one skilled in the art.

Solvates of SCD1 inhibitor include, for example, those made with solvents that are pharmaceutically acceptable. Examples of such solvents include water (resulting solvate is called a hydrate) and ethanol and the like. Suitable solvents are physiologically tolerable at the dosage administered.

In embodiments of the present application, the SCD1 inhibitor described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

The SCD1 inhibitors may also exist in different tautomeric forms and it is intended that any tautomeric forms which the compounds form, as well as mixtures thereof, are included within the scope of the present application.

The SCD1 inhibitors may further exist in varying polymorphic forms and it is contemplated that any polymorphs, or mixtures thereof, which form are included within the scope of the present application.

The SCD1 inhibitors may further be radiolabeled and accordingly all radiolabeled versions of IMPDH inhibitors are included within the scope of the present application. The IMPDH inhibitors also include those in which one or more radioactive atoms are incorporated within their structure.

The SCD1 inhibitors are suitably formulated into pharmaceutical compositions for administration to subjects in a biologically compatible form suitable for administration in vivo. Accordingly, the present application further includes a pharmaceutical composition comprising an effective amount of a SCD1 inhibitor and a pharmaceutically acceptable carrier wherein the SCD1 inhibitor is present in amount effective to treat medulloblastoma (optionally, high risk medulloblastoma).

The SCD1 inhibitors are administered to a subject in a variety of forms depending on the selected route of administration, as will be understood by those skilled in the art. For example, a SCD1 inhibitor is administered by oral, inhalation, parenteral, buccal, sublingual, nasal, rectal, vaginal, patch, pump, minipump, topical or transdermal administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

Parenteral administration includes systemic delivery routes other than the gastrointestinal (GI) tract, and includes, for example intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

In some embodiments, a SCD1 inhibitor is orally administered, for example, with an inert diluent or with an assimilable edible carrier, or it is enclosed in hard or soft shell gelatin capsules, or it is compressed into tablets, or it is incorporated directly with the food of the diet. In some embodiments, the SCD1 inhibitor is incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release ortimed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example as liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine or phosphatidylcholines. For oral administration in a capsule form, useful carriers or diluents include lactose and dried corn starch.

In some embodiments, liquid preparations for oral administration take the form of, for example, solutions, syrups or suspensions, or they are suitably presented as a dry product for constitution with water or other suitable vehicle before use. When aqueous suspensions and/or emulsions are administered orally, the SCD1 inhibitor is suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry a SCD1 inhibitor and use the lyophilizates obtained, for example, for the preparation of products for injection.

In some embodiments, a SCD1 inhibitor is administered parenterally. For example, solutions of a SCD1 inhibitor are prepared in water suitably mixed with a surfactant such as hydroxypropylcellulose. In some embodiments, dispersions are prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the SCD1 inhibitor are usually prepared, and the pH's of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiment, such compositions include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

In some embodiments, a SCD1 inhibitor is formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions in oily or aqueous vehicles, and contain formulating agents such as suspending, stabilizing and/or dispersing agents. In all cases, the form must be sterile and must be fluid to the extent that easy syringability exists. Alternatively, the SCD1 inhibitors are suitably in a sterile powder form for reconstitution with a suitable vehicle, e.g., sterile pyrogen-free water, before use.

In some embodiments, compositions for nasal administration are conveniently formulated as aerosols, drops, gels and powders. For intranasal administration or administration by inhalation, the SCD1 inhibitors are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the SCD1 inhibitor. Capsules and cartridges (made, for example, from gelatin) for use in an inhaler or insufflator are, for example, formulated containing a powder mix of a SCD1 inhibitor and a suitable powder base such as lactose or starch. The aerosol dosage forms can also take the form of a pump-atomizer.

Compositions suitable for buccal or sublingual administration include tablets, lozenges, and pastilles, wherein a SCD1 inhibitor is formulated with a carrier such as sugar, acacia, tragacanth, or gelatin and glycerine. Compositions for rectal administration are conveniently in the form of suppositories containing a conventional suppository base such as cocoa butter.

Suppository forms of the SCD1 inhibitors are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, PA, 1980, pp. 1530-1533 for further discussion of suppository dosage forms.

In some embodiments, a SCD1 inhibitor is coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, in some embodiments, a SCD1 inhibitor is coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

In some embodiments, a SCD1 inhibitor may be coupled with viral, non-viral or other vectors. Viral vectors may include retrovirus, lentivirus, adenovirus, herpesvirus, poxvirus, alphavirus, vaccinia virus or adeno-associated viruses. Non-viral vectors may include nanoparticles, cationic lipids, cationic polymers, metallic nanoparticles, nanorods, liposomes, micelles, microbubbles, cell-penetrating peptides, or lipospheres. Nanoparticles may include silica, lipid, carbohydrate, or other pharmaceutically acceptable polymers.

In an embodiment, the SCD1 inhibitor can be administered by intraperitoneal (IP) injections, intranasally, intracranially, intravenously, or orally.

A SCD1 inhibitor including pharmaceutically acceptable salts and/or solvates thereof is suitably used on their own but will generally be administered in the form of a pharmaceutical composition in which the SCD1 inhibitor (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient, and from about 1 wt % to about 99.95 wt % or about 30 wt % to about 99.90 wt % of a pharmaceutically acceptable carrier, all percentages by weight being based on the total composition.

### Examples

The following non-limiting examples are illustrative of the present disclosure:

### Methods

Cell Culture: SU_MB002, HD-MB03, MED411FHTC (MYC-amplified G3-MB PDOX) and hNSC197 were all cultured in Neurocult™ Complete (NCC) media (Stem Cell Technologies, #05751), consisting of NeuroCult NS-A Basal Medium, Neurocult Supplement, epidermal growth factor (20 ng/mL), fibroblast growth factor (10 ng/mL), 0.1% heparin and 1% penicillin-streptomycin. HD-MB03 NCC media was supplemented with 10% FBS, and hNSC197 were cultured on Poly-L-Ornithine+Laminin coated plates. Each cell line was conditioned in NCC only for minimally 24 hours prior to an experiment. SU_MB002 was derived at recurrence, the patient received cyclophosphamide only and was kindly gifted by Dr. Yoon-Jae Cho. HD-MB03 was isolated from a patient with metastasized G3-MB and is treatment naïve and kindly gifted by Dr. Till Milde. MED411FHTC was purchased from Dr. Olson's laboratory.

Lipidomics: All cell lines were cultured in the same media conditions (NeuroCult Complete) for 24-48 hours prior to collection for lipidomics. Cells were pelleted, 3,000,000 per cell line in triplicates, and washed with PBS and stored at −80° C. until processed. Lipid extraction and untargeted analysis was performed by Cayman Chemical Company, as described below. Briefly, Lipid extraction was performed by means of methyl-tert-butyl ether (MTBE)-based liquid extraction protocol. After thawing, 200 μL PBS was added to each sample. Next samples were vortexed and transferred to 8 m: screw-cap tubes containing 600 μL methanol 10 μL custom internal standard mix and 4 mL MTBE. For the Neurocult Complete cell culturing control sample, 200 μL was transferred directly to an 8 mL screw-cap tube containing 1.2 mL methanol, 10 μL custom internal standard mix, and 4 mL MTBE. After vortexing all samples. The samples were incubated on a tabletop shaker for 1 hour (500 rpm, RT). Next, 1 mL of water was added to each sample, which were subsequently vortexed and centrifuged (2500×g, 5 min) to induce phase separation. The upper organic phase of each sample was carefully removed using a Pasteur pipette and collected into a clean glass tube. The remaining aqueous phase was reextracted with 2.5 mL of the upper phase of a mixture MTBE/methanol/water 10:3:2.5 (v/v/v). After vortexing and centrifuging, the organic phase was collected and combined with the initial organic phase. The extracted lipids were dried overnight in a SpeedVac® vacuum concentrator. The dried lipid extracts were reconstituted in 200 μL n-butanol/methanol, 1:1 (v/v) and transferred into autosampler vials for analysis by LC-MS/MS. Quality control sample aliquots, derived from 20 μL human plasma and processed as described above, were continuously injected to assess LC-MS performance. Lipostar® software (version 2.1.4b5, Molecular Discovery) was utilized for feature detection, noise and artifact reduction, peak alignment, normalization, and lipid identification by querying the Lipid Maps database.

In vitro functional BTIC assays: For the self-renewal assay, cells were plated at a density of 200 cells/well in a 96wp in quadruplicates (200 μL/well). Cells were incubated for a period of 72 hrs, after which sphere counts were performed manually with a light microscope (10× magnification). Exclusion criteria were less than 7 healthy cells per sphere. In Oleic Acid (OA). rescue experiments, cells were seeded into 96wps with NCC supplemented with 50 μM OA.

In vitro drug studies/preparation: For drug assays, cells were plated at a density of 1000 cell/well in a 96wp and incubated for 2 h hours. Next, 10 mM drug stocks (DMSO) were serially diluted (3fold dilution) and pipetted into wells (1 μL/well) to achieve nine final volumes starting from 25 μM range (in triplicates). Cell viability was assessed after 72 hrs. PrestoBlue® (ThermoFisher™ Scientific; Catalogue #A13261) was added (20 μL/well) and fluorescence was measured after 4 hrs of incubation using a FLUOstar® Omega Fluorescence 556 Microplate reader (BMG LABTECH) at excitation and emission wavelengths of 535 nm and 600 nm, respectively. Readings were analyzed using Omega® analysis software. Oleic Acid (OA) rescue experiments were performed by seeding cell into the 96 plates in NCC supplemented with 50 μM OA.

Cloning CRISPR-Cas9 constructs: All sgRNA sequences were cloned into the lentiCRISPRv2 plasmid as previously described and successful integration was subsequently confirmed by Sanger sequencing⁸⁵. CRISPR data analysis was performed as previously described⁴³.

Western blotting: Protein lysates were derived from 500,000 to 1,000,000 cells. Cells were pelleted, washed with PBS and subsequently lysed with RIPA buffer containing HALT protease inhibitor cocktail (1:100) (ThermoFisher). The Bradford assay (BioRad™, Cat #5000112) was utilized to determine lysate protein concentrations. Western blot protein samples were prepared (30 μg/sample), boiled for 5 minutes and loaded on SDS-PAGE gels. Samples were separated by size by means of gel electroporation and then transferred to PVDF by wet transfer. A specific binding was blocked by submerging PVDF membranes in MeOH for 30 seconds and allowed to dry at RT. Once fully dried, membranes were probed with primary antibodies Rabbit polyclonal anti-SCD1 (Cell Signaling, #2438) was used at a 1:1000 dilution, mouse monoclonal anti-Vinculin (Sigma-Aldrich, MAB3574) was used at a 1:10,000 dilution. Horse-radish peroxidase-conjugated Secondary antibody anti-rabbit (Biorad, CAT #170-6515) and anti-mouse (Cell Signaling, CAT #7076) were used with Super Signal™ West Femto Maximum Sensitivity Substrate (Thermo Scientific, 34095) and Biorad Clarity ECL reagents respectively for band development. Protein band quantification was performed by analyzing mean pixel density (ImageJ) and subsequent normalization of target protein to Vinculin control.

Proteome profiler array: Proteome profiler array (R&D Systems, ARY003C) was performed according to manufacturer's guidelines (330 μg per sample). Briefly, cells were collected on day 4 after transduction (2,300,000 cells/sample), washed with PBS and pelleted. PBS supernatant was aspirated from cell pellets and cell pellets were stored at −80° C. until processed. Cells were lysed according to manufacturer's guidelines and protein quantity was determined with a Bradford assay (BioRad®, Cat #5000112). Phospho-Kinase Array membranes were imaged on a ChemiDoc® (Biorad) and protein quantification was performed with ImageJ.

Generation of SCD1 single clone KO lines: Cells were transduced with LentiCRISPRv3-SCD1 guide 4. pooled SCD1 KO G3-MB cells were puromycin selected and subsequently flow sorted for GFP expression and high cell viability. Single cells (GFP+, high viability) were seeded into wells of a 96 well plate (one clone per well) containing 200 μL NCC, supplemented with 12.5 μM Oleic Acid (Thermofisher, Cat. No: 031997.06).

In vivo studies: For the intracranial model, cells were collected in PBS kept on ice until injections. PDOX were generated by intracranial injections of 10,000 cells in 10 μL PBS per mouse. For the Flank model cells were collected in a 1:1 mixture of PBS and Matrigel and kept on ice until injection. Mice were shaved at the flank and subcutaneously injected with 2,000,000 cells in 100 μL PBS/Matrigel. For preclinical studies cells were transduced to express enhanced firefly luciferase (FFLUC) to enable IVIS imaging. CAY10566 (10% DMSO, 40% PEG300, 5% Tween80, 45% saline) was administered by intraperitoneal (IP) injections 20 mg/kg.

In Vivo Imaging systems (IVIS) imaging: Mice were sedated by isoflurane gas and subsequently subcutaneously injected with 150 mg/kg (in PBS) luciferin substrate. After a 10 min incubation period, bioluminescent signal (photons per second, p/s) was detected by In Vivo Imaging systems (IVIS) and quantified with Living Image software.

Immunohistochemistry: Mouse brains were fixated in 10% formalin for 5 days. Afterwards fixed brains were washed with 50% EtOH and stored in 70% EtOH at 4° C. Next brains were sliced and Paraffin-embedded. FFPE tissues were sectioned onto slides and deparaffinized. Samples were stained with H&E.

Cell cycle and apoptosis with FACS: Apoptotic cells were detected via FITC Annexin V/Pi staining (Thermo Fisher Scientific; Catalogue #A23204). Briefly, 100,000 cells per sample were washed twice and resuspended in 100 μL binding buffer (Thermo Fisher Scientific; Catalogue #V13246). Next samples were incubated with Annexin V/Pi and vortexed. Samples were incubated for 15 minutes in the dark, 400 μL binding buffer was added, and samples were analyzed. Cell cycle stages were detected by following the Coulter DNA Prep Reagents Kit (Beckman Coulter; Catalogue #6607055). Briefly, 200,000 cells per sample in 50 μL PBS were incubated in 50 μL DNA PREP LPR, vortexed for 10 s and subsequently incubated with 750 μL of DNA Prep Stain. Samples were vortexed for 10 s and incubated at RT for 15 min, after which samples were analyzed. Flow cytometry analysis was performed with the CytoFLEX LX flow cytometer.

### Bioinformatics Analyses

Lipidomics: differential abundance and composition map: Following lipidomic analysis obtained from Cayman, the dataset of detected lipids and abundance (Area Ratio Analyte/Internal Standard) for each sample in triplicates was further processed. Internal standards were removed, and the maximum value obtained from the blank or the NCC media sample for each lipid (background) was subtracted from all samples. Negative values were replaced by 0, and those lipids containing 0 value for all samples were removed. In order to perform statistical analysis, zeros were substituted with half of the minimum value detected in the dataset. To identify lipids that are differentially abundant between NSC and MB-G3 samples, T-test comparison (for equal or not equal variances depending on F-test results) for all lipids was performed. Results were corrected for multiple testing using false discovery rate (FDR). Fold change (FC) and log 2FC were calculated. To identify differentially abundant lipids, a threshold of logFC>|2|(log 2FC>|1|), and p-corrected values<0.05 was considered for statistical significance. To investigate the composition of fatty acids in TGs, the table was subset for the lipid species of interest and the average abundance from each sample triplicates was calculated. The lipid abundance and composition (sum saturation level and fatty acyl chain length) were visualized relative to each sample. Analyses were performed using R (v. 4.2.2) and visualized using the packages pheatmap, ggplot2 and viridis.

RNA sequencing: 300,000 Cells were treated with 4 nM CAY10566 or DMSO for 72 hrs. Afterwards, total RNA was isolated purified with and subsequently send for Ilumina NextSeq 2000 sequencing. FASTQ files were loaded to SHARCNET (Graham supercomputer), and sequencing reads were aligned to the Ensembl human reference genome (GRC38) using STAR aligner. FastQC and MultiQC analysis were performed. Normalization of expression levels was performed using CPM and TMM methods and visualization of the data was performed in R using MA plot, RLE plot, PCA and heatmap with hierarchical clustering; TMM normalization method was selected (R v4.2.2).

Differential expression analysis: From the RNA-seq data, raw read counts less than 5 were filtered prior to performing differential expression analysis using DESeq2. Identification of differential expressed genes (DEGs) was based on adjusted p-value<0.05 and log 2FC>111. Data from 763 pediatric MB patients from the Cavalli dataset available in the GEO repository (GSE85217) was analyzed. Differential expression analysis was performed using limma. The threshold to identify DEGs was adjusted p-value<0.01 and log 2FC>|1|. Visualization of DEGs was performed using a volcano plot and heatmap with hierarchical clustering (R v4.2.2).

Gene set enrichment analysis: DEGs were annotated and gene set enrichment analysis was calculated and visualized using R (R packages: annotate, org.Hs.eg.db, clusterProfiler, GOstats, GO.db) and using the bioinformatic web server Enrichr®⁸⁻⁸⁸. Databases used were: KEGG 2021, GSEA MSigDB Hallmarks of Cancer 2020,⁸⁹⁻⁹¹. Statistically significant enriched gene sets were considered as adjusted p-value <0.05 (5% FDR). In a specific analysis, a 10% FDR was used.

Survival analysis: Survival analysis was performed using quartiles distribution from each molecular subgroup as thresholds to subset patients into very low/very high SCD expression (<Q1 and >Q3). Survival analysis was performed with 612/763 patient samples from the Cavalli dataset, after removing samples with NA info in Event (0,1) and/or survival(time). The Cox regression model for survival analysis was used and p-values, Hazard-Ratio and 95% confidence interval were calculated. Overall survival was visualized using Kaplan-Meier curves. R packages: survival, gtsummary.

Correlation analysis: The distribution of SCD expression data was visualized with a density plot and performing a Q-Q plot (quantile-quantile plot) (ggpubr) and performing a Shapiro test, to determine whether the data followed a normal distribution. SDC expression followed a normal distribution (p-value>0.05), so parametric statistical tests were used. Pearson correlation method was used as SCD and MYC expression are continuous variables and Spearman and Kendall methods are for rank correlations. However, MYC expression did not present a normal distribution (Shapiro test p-value<0.05).

All Bioinformatic analyses were performed using R version 4.2.2.

Statistical Analysis: Experiments were performed twice to ensure reproducibility. A minimum of four technical replicates were used for mean comparisons. Differences between two groups were measured via two-tailed unpaired t-tests. More than two groups were compared via one-way ANOVA analysis followed by appropriate multiple comparisons. Two-way ANOVA analysis followed by appropriate multiple comparisons were used when comparing more than 2 groups to at least 2 variables. In vivo Kaplan-meier survival data were measured for significance using the Log-rank test.

Ethics Approvals: All animal experiments were performed in accordance with the Canadian Council on Animal Care (CCAC) under animal utilization protocol (22-12-38) and was approved by the Animal Research Ethics Board (AREB). The isolation of human tissues was performed using protocols approved by the Human Integrated Research Ethics Board (HIREB).

### Results and Discussion

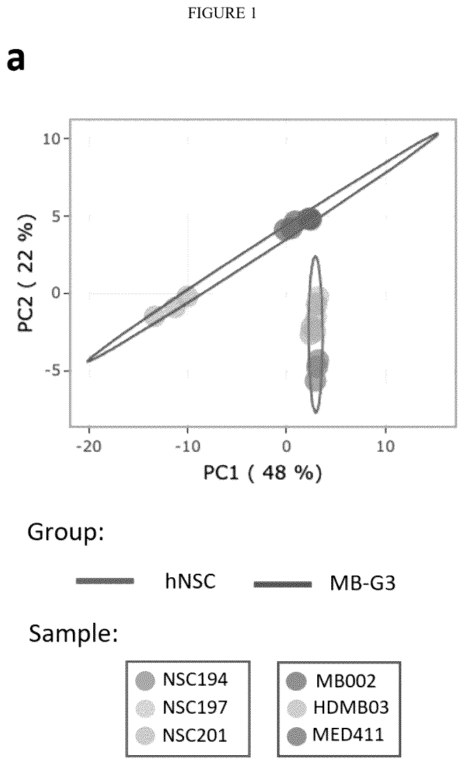

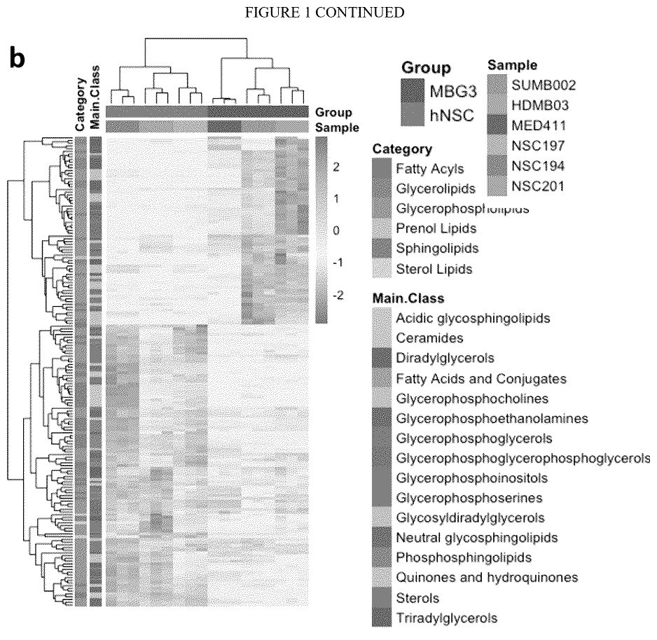

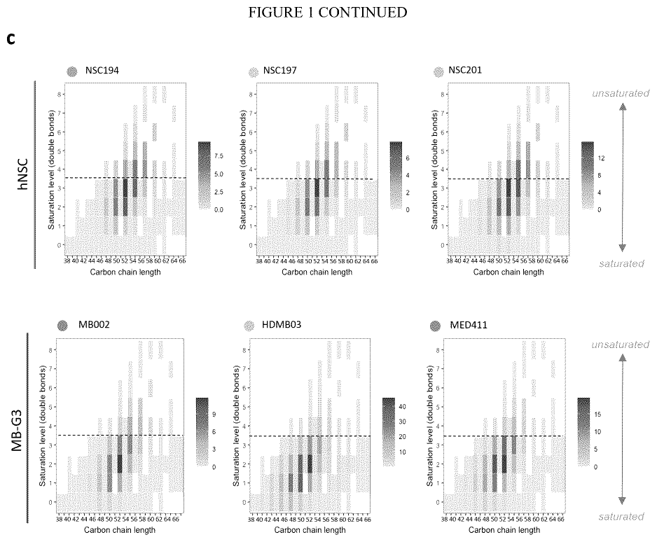

Lipidomic analysis reveals differential abundance of unsaturated FAs in G3-MB: To explore the MYC-driven G3-MB lipidome and to identify therapeutically targetable sensitivities of lipid bio-production, untargeted liquid chromatography-mass spectrometry (LC-MS) analysis was employed (performed at Cayman Chemical) and compared three MYC-driven G3-MB patient-derived lines (SU_MB002, HD-MB03 and MED411FHTC) to three hNSC cell lines (hNSC194, hNSC197 and hNSC201FT), the cell-of-origin44. Principal Component Analysis (PCA) revealed two distinct clusters specific to the MYC-driven G3-MB and the NSC samples (FIG. 1 a). 172/680 lipids were detected with significantly different abundance between NSCs and MYC-driven G3-MB (adjusted p-value <0.05 and log 2FC>|1|). These differentially abundant lipids consist of fatty acyls, glycerolipids, glycerophospholipids, prenol lipids, sphingolipids and sterol lipids (FIG. 1 b).

Given the high FA demand of cancer cells for survival¹², the composition of 99 TGs (consisting of one glycerol and three FAs) was investigated, focusing on FA carbon chain length and number of double bonds. Interestingly, all MYC-driven G3-MB patient-derived lines consistently displayed a decreased abundance of desaturated TG species when compared to the NSC lines (FIG. 1 c). Taken together, these data indicate that MYC-driven G3-MB and NSCs differ substantially in abundance of lipid species and saturation levels of FAs.

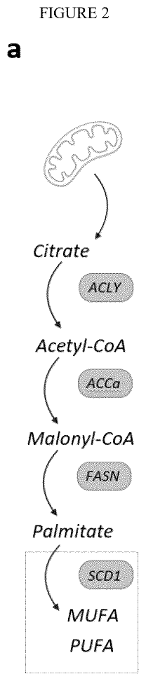

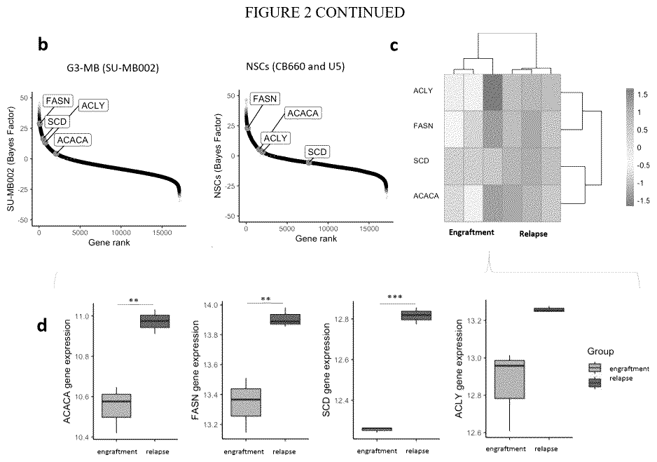

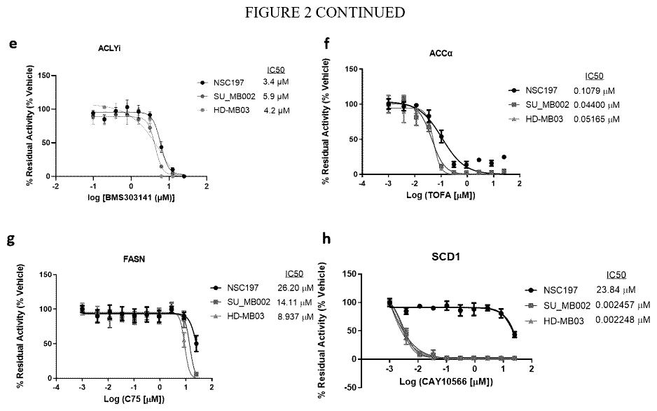

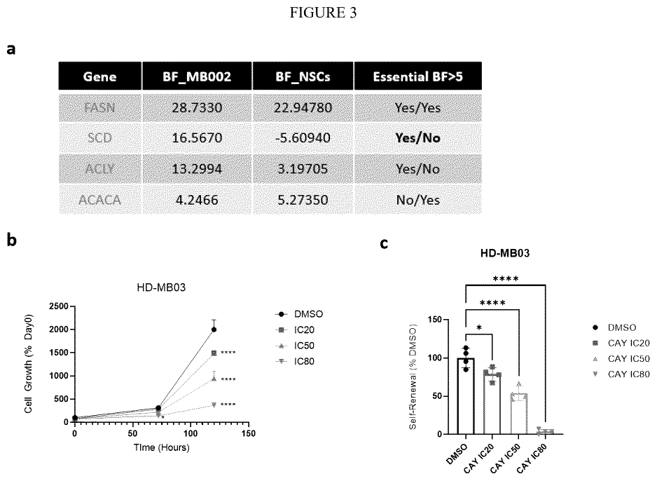

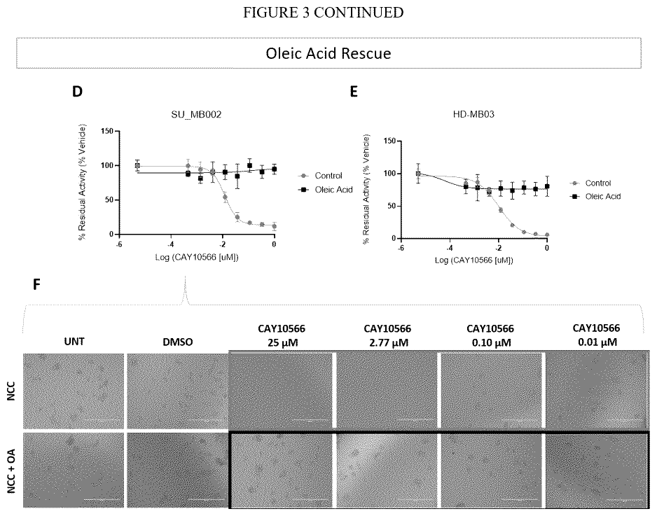

SCD1 is essential in medulloblastoma and is a potent therapeutic target in vitro: Considering the discrepancy in FA saturation between the G3-MB and NSC lipidomes, the cytoprotective role of lipid desaturation⁴⁵ and the fact that DNL is downstream of MYC⁴⁶, it was hypothesized that targeting the de novo lipid synthesis (DNL) pathway may be a therapeutic vulnerability in MYC-amplified G3-MB. To confirm this hypothesis, the datasets of the previously conducted genome-wide CRISPR-Cas9 TKOv3 loss-of-function screen in SU_MB002⁴³ (FIG. 2 a, b and FIG. 3 a) and murine PDX derived SoC adapted transcriptomic HD-MB03 model⁹ were probed (FIG. 2 c, d).

When probing the SU_MB002 CRISPR KO dataset and comparing these findings with a publicly available CRISPR KO screen in NSCs 47, SCD was found to be the most essential gene (of the DNL pathway) in MBs but not in NSCs (MB-BF=16.6, NSC-BF=−5.6), followed by ACLY (MB-BF=13.3 and NSC-BF=3.2) (FIG. 2 b and FIG. 3 a). These findings suggest that SCD (and ACLY) may represent cancer-selective vulnerabilities in MB.

After confirming DNL gene essentiality in G3-MB, transcriptomic expression of DNL enzymes upon orthotopic engraftment was compared to treatment relapse in the murine PDX derived SoC adapted model. Interestingly, expression of all four genes increased at relapse, with SCD, FASN and ACACA having a statistically significant increase in expression (FIG. 2 c, d). Together, these data indicate that the DNL enzymes, particularly SCD1, may be a targetable vulnerability only in G3-MB, while sparing healthy NSC populations.

The DNL pathway was explored as a therapeutic vulnerability by performing an in vitro small molecule inhibitor screen in two G3-MB lines (SU_MB002 and HD-MB03) and the control hNSC197 patient derived cell line. The ACLY inhibitor BMS303141 (FIG. 2 e), the ACCα inhibitor TOFA (FIG. 2 f), the FASN inhibitor C75 (FIG. 2 g) and the SCD1 inhibitor CAY10566 were screened (FIG. 2 h). Strikingly, CAY10566 was found to have a potent 10,000-fold therapeutic window for SU_MB002 and HD-MB03 cells (IC50s at 2.45 nM and 2.24 nM respectively) when compared to control NSC197 at IC50 of 23.84 μM (FIG. 2 h).

Considering this potent therapeutic window, the cytotoxic effect of CAY10566 was further characterized and it was found that CAY10566 significantly impaired G3-MB cell growth (FIG. 3 b) and self-renewal (FIG. 3 c) in a dose-dependent manner. Importantly, the CAY10566-induced cytotoxic effects were reversed by supplementing cell culturing media with Oleic Acid (OA), an enzymatic product of SCD1, in both SU_MB002 and HD-MB03, indicating on-target induced cell death (FIG. 3 d-f). Additionally, the inhibitory effect of CAY10566 on G3-MB self-renewal suggests that SCD1 is a regulator of stemness in G3-MB.

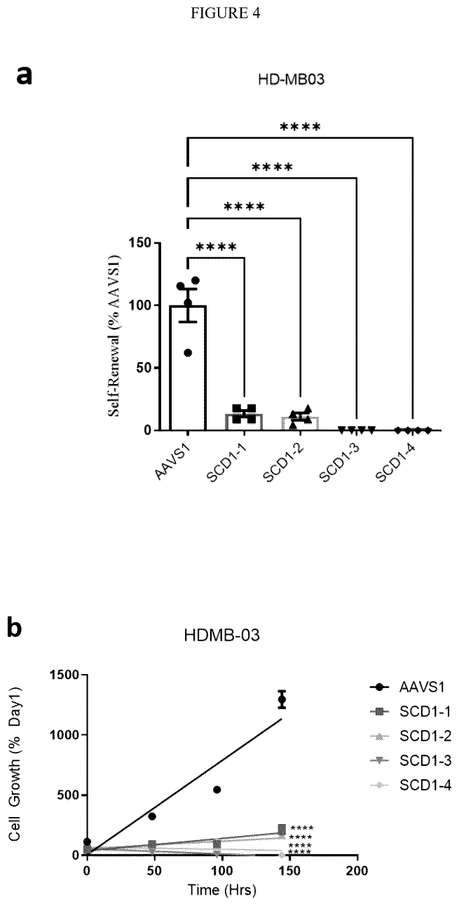

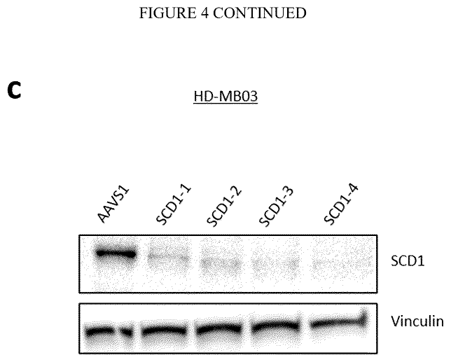

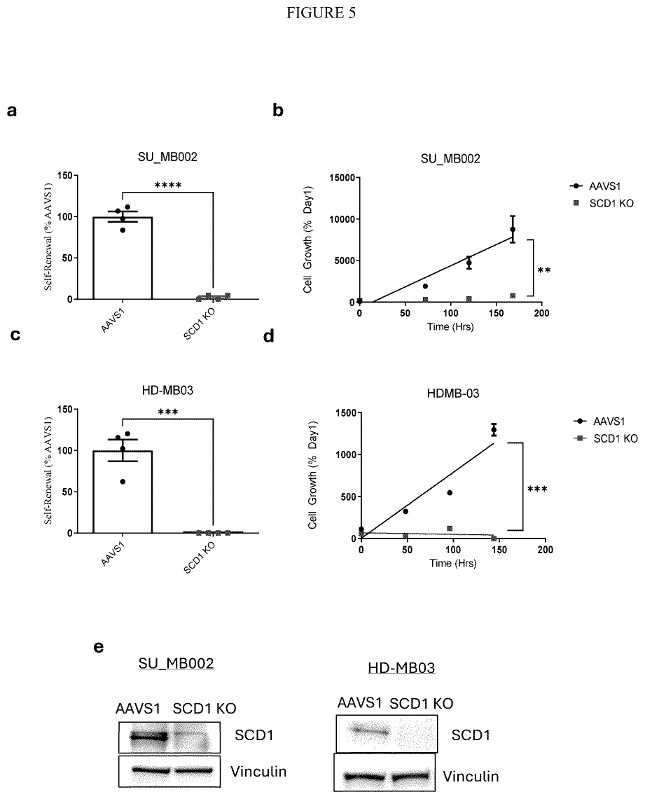

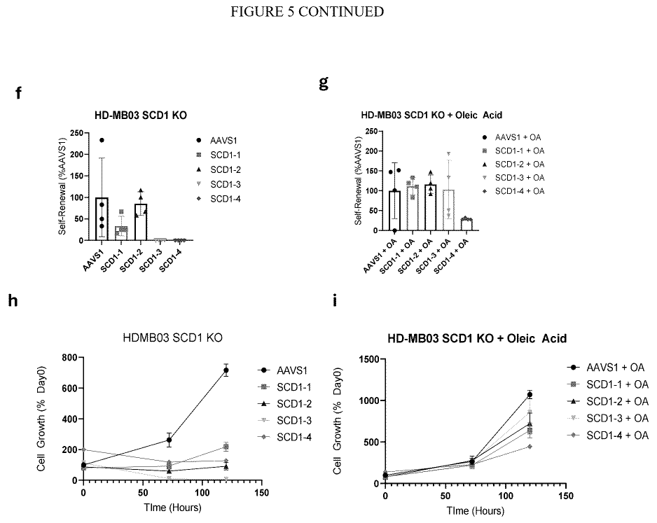

Genetic knock-Out phenocopies functional effects of small molecule SCD1 inhibition: The role of SCD1 was investigated as a regulator of stemness by subjecting SU_MB002 & HD-MB03 SCD1 KO cells to self-renewal and cell growth assays. To this end, four SCD1 sgRNA guides in HD-MB03 were compared and sgSCD1 guide number four (SCD1-4) was selected for further experiments (FIG. 4 a-c). Genetic KO of SCD1 completely ablates the sphere-forming capacity and the proliferative capacity of SU_MB002 (FIG. 5a,b) and HD-MB03 (FIG. 5 c, d). Western Blots confirming SCD1 KO are displayed in FIG. 5e. Additionally, media supplementation with OA rescued SCD1 KO inhibitory effects on self-renewal (FIG. 5 f,g) and proliferation (FIG. 5 h, i), again confirming on-target KO effects.

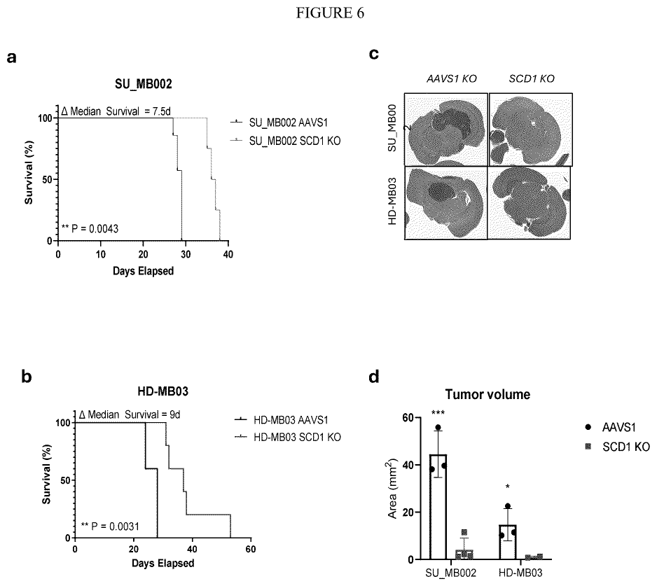

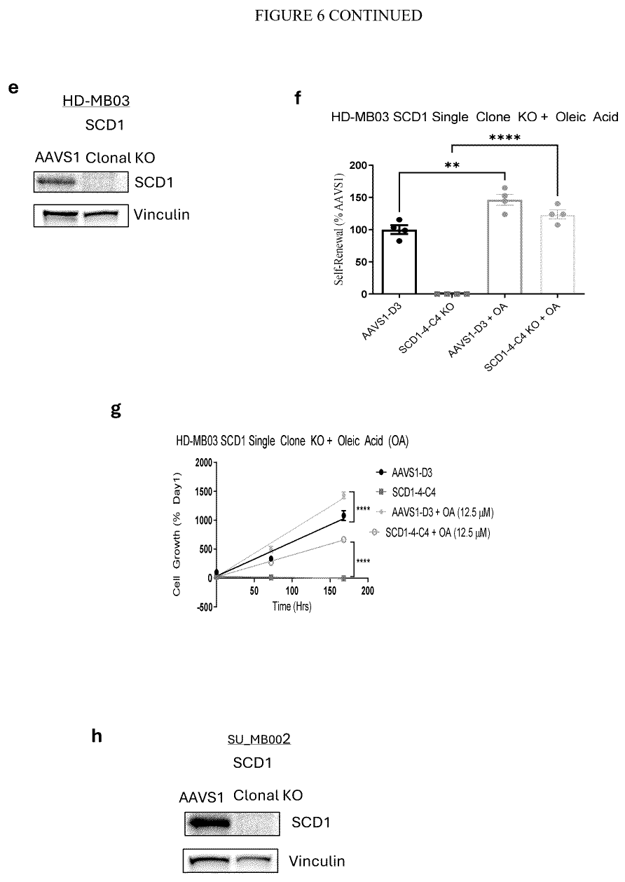

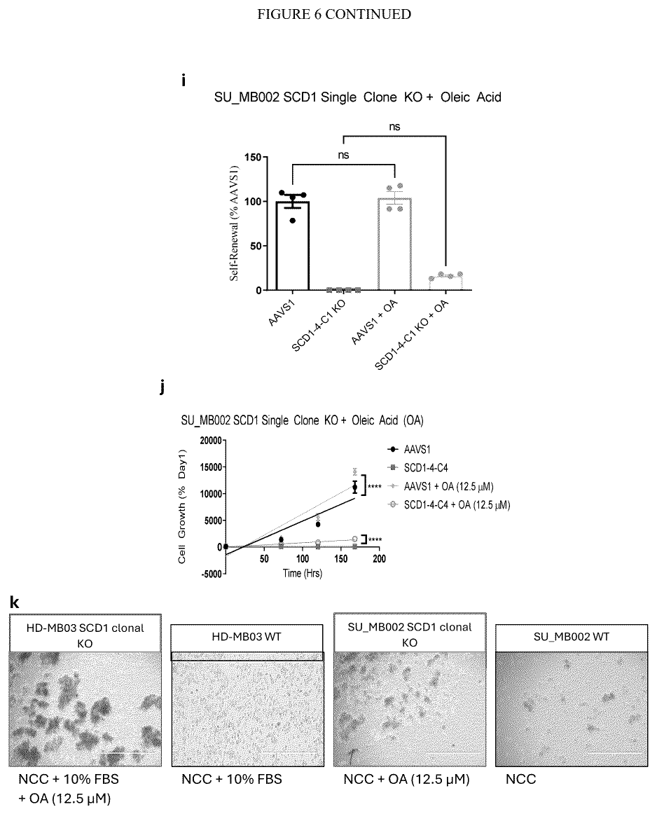

Genetic and pharmacological inhibition of SCD1 results in increased survival in a PDOX adapted mouse model: The in vitro results identify SCD1 as a regulator of sternness in G3-MB and a potential therapeutic target⁵. In order to confirm a loss of BTIC frequency after SCD1 KO, the gold standard in vivo assay of stemness⁴ was utilized. To this end, equal numbers of SCD1 KO or AAVS1 KO (pooled) tumor cells were intracranially injected in NSG mice and monitored survival and tumor progression. SCD1 KO significantly increased mouse survival in both SU_MB002 (extended median survival=7.5 days, p=0.0043) and HD-MB03 (extended survival=9 days, p=0.0305) engrafted mice (FIG. 6 a, b). Histological examination of time-matched brain tissues revealed a significant brain tumor burden reduction in the SCD1 KO injected mice when compared to AAVS1 KO control injected cells (FIG. 6 c, d).

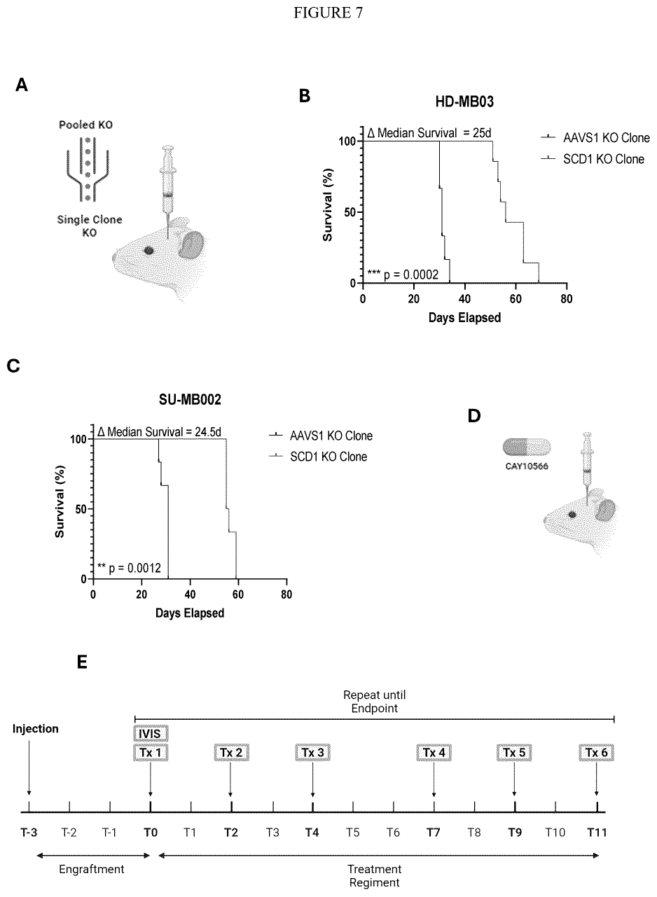

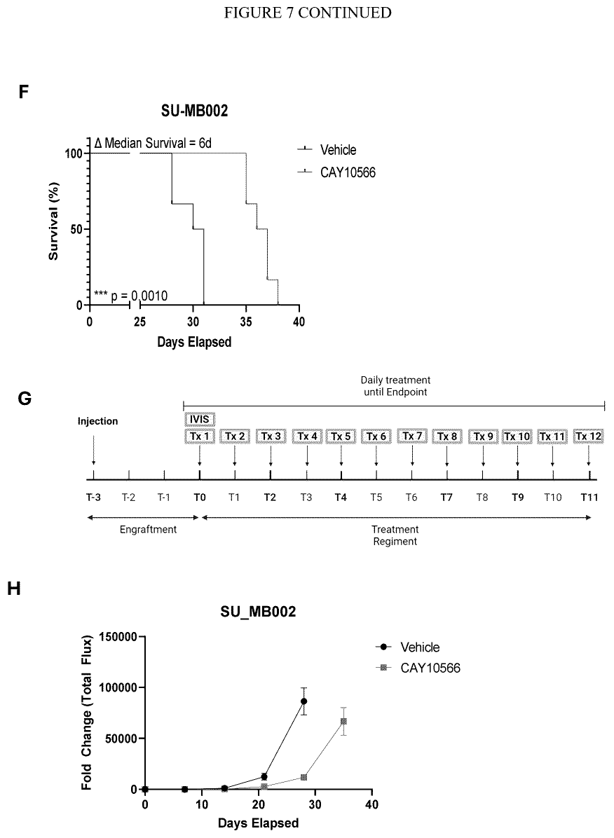

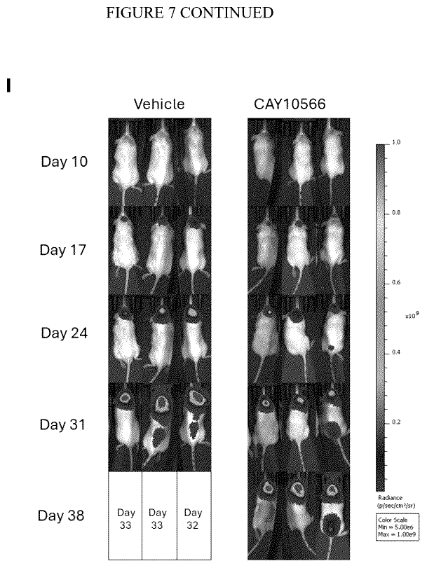

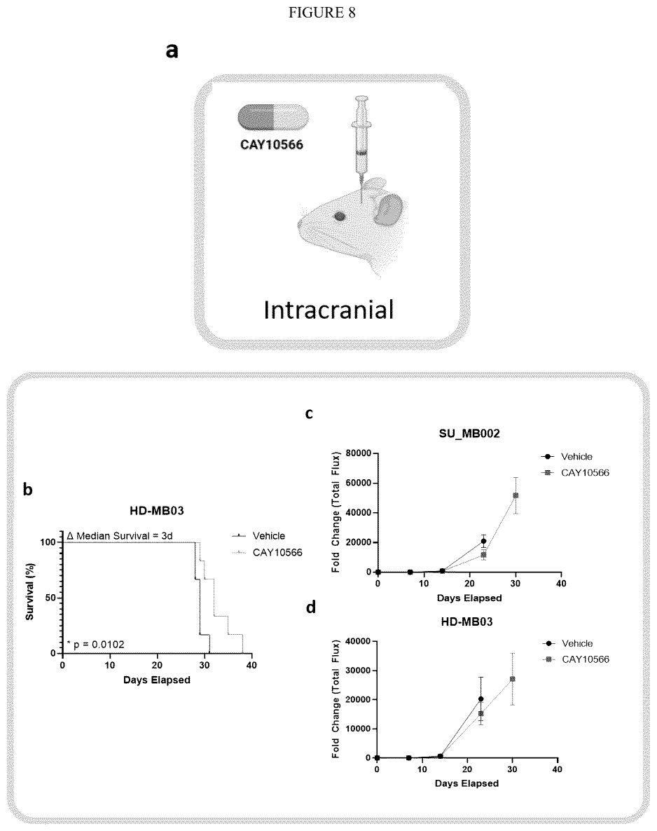

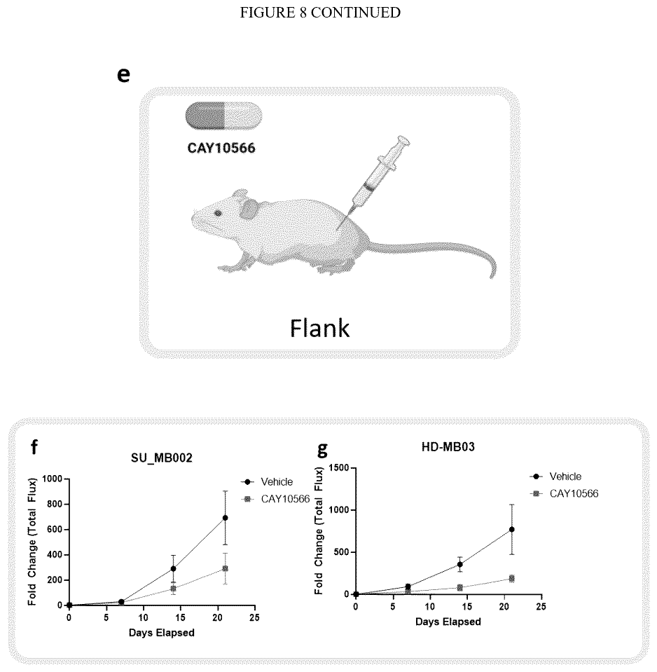

Surprisingly, mice engrafted with SCD1 KO G3-MB cells eventually succumb to tumor progression and tumor burden. Attributing these mortalities to a pooled KO effect, where residual wildtype (WT) cells eventually overtake the tumor mass, clonal SCD1 KO G3-MB lines were generated by FLOW sorting for GFP and by leveraging OA-mediated rescue of cell viability after SCD1 KO. Loss of SCD1 protein was confirmed by Western Blot (FIG. 6 e, h). Next, SU_MB002 and HD-MB03 SCD1 single clones were injected intracranially in mice and monitored survival overtime (FIG. 7 a-c). Intracranial injection of SCD1 single clone KO cells in NSG mice greatly improved survival outcome when compared to pooled KO's, extending survival well beyond the G3-MB WT mouse model's survival limit (SU_MB002 clonal SCD1 median survival increase=24.5 days and HD-MB03 clonal SCD1 median survival increase=25 days) (FIG. 7 b, c). To confirm clonal function, clonal cells of the same population as the intracranially injected cells were subjected to in vitro self-renewal and cell growth assays supplemented with OA in parallel to the in vivo study. As expected, supplementation with OA significantly increased cell growth and self-renewal capacity of HD-MB03 and SU_MB002 AAVS1 single clone controls when compared to WT HD-MB03 and SU_MB002 cells. (FIG. 6 e-j) Clonal HD-MB03 SCD1 KO cell growth significantly increased, reaching almost the same growth rate as AAVS1 controls (FIG. 6g) and OA supplementation significantly increased SU_MB002 SCD1 clonal KO cells, however to a lesser extent (FIG. 6 j). Moreover, OA supplementation had similar restorative effects in self-renewal capacity, with full rescue in HD-MB03 SCD1 clones and marginally restored function in SU_MB002 SCD1 clonal KO cells (FIG. 8 f, j). Clonal KO cells underwent remarkable phenotypic changes when compared to WT cells and these are displayed in (FIG. 6 k). The marginal effect of OA-mediated rescue in SU_MB002 clonal SCD1 KO cells when compared to HD-MB03 clonal SCD1 KO cells can be explained by cell membrane integrity³⁸. SU_MB002 WT cells have poor membrane integrity, and media substitution with a single FA species (OA) does not fully supplement loss of MUFA and PUFA species after SCD1 KO, rendering cell membrane integrity even weaker than their WT counterparts. Additionally, SU_MB002 cells are not cultured in the presence of FBS, as HD-MB03 cells are. The presence of FBS during culturing may give HD-MB03 cells an advantage in terms of lipid availability and thus cell membrane integrity during these functional assays²¹. The combination of differential membrane integrity and FBS supplementation thus explains the differences between SU_MB002 and HD-MB03 SCD1 clonal KO cells in the functional assays.

Next, the efficacy of CAY10566 treatment of G3-MB was tested in vivo (FIG. 7 d-i). Mice were intracranially injected with SU_MB002 or HD-MB03 cells expressing firefly luciferase (FFLUC) cells. Treatment was initiated three days post-injection and consisted of CAY10566 administration via IP injections (20 mg/kg CAY10566). IP injections were given three days a week (Monday, Wednesday, and Friday) until endpoint and tumor progression was monitored by IVIS once weekly (FIG. 7 e). Treatment significantly increased survival for both SU-MB002 (extended survival=6 days, p=0.0010) and HD-MB03 (extended survival=3 days, p=0.0102) engrafted mice (FIG. 7 f and FIG. 8 b), however no detectable difference in tumor growth was observed by IVIS (FIG. 8 c, d). Considering CAY10566's limited blood brain barrier (BBB) penetrability⁴⁸, the treatment regimen was repeated in a flank model in order to bypass the BBB (FIG. 8 e-g). With the flank model a decrease in tumor growth by IVIS was observed in both SU_MB002 and HD-MB03 bearing mice (FIG. 8 f, g), indicating the BBB was a limiting factor within the intracranial model and more efficient BBB penetrable SCD1 inhibitors are warranted. Finally, the CAY10566 IP regimen was optimized by increasing treatments from three days per week, to daily CAY10566 IP treatments to overcome the limited BBB permeability (FIG. 7 e). Notably, the increased dosing led to a reduction in tumor growth and significant survival advantage that was detectable by IVIS despite the BBB bottleneck (FIG. 7 h, i). These results confirm SCD1 as a potent therapeutic target for MYC amplified G3-MB.

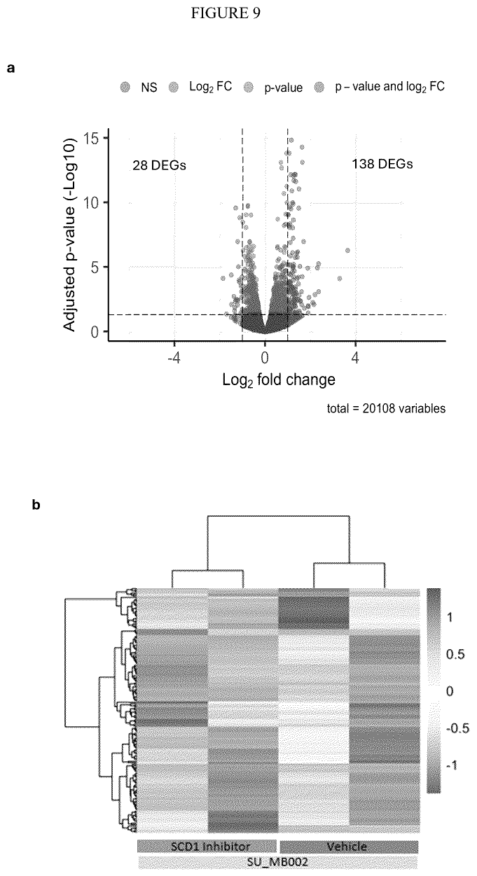

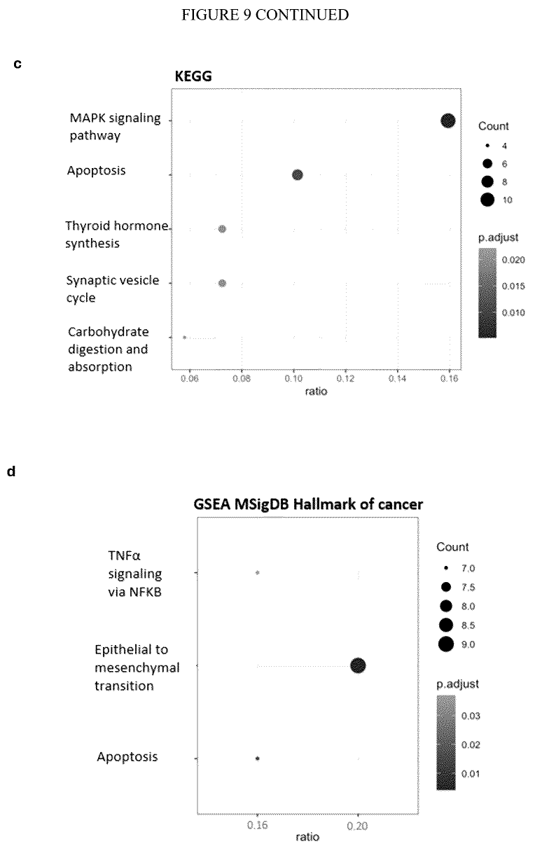

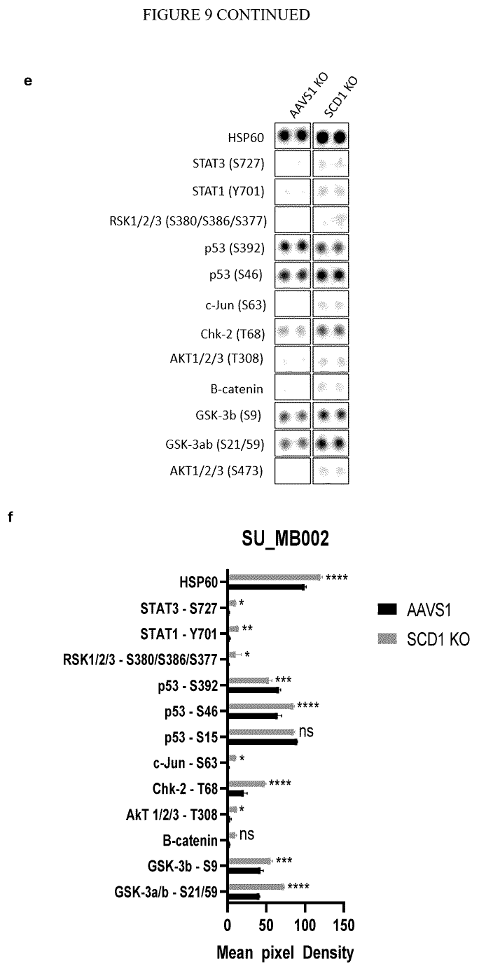

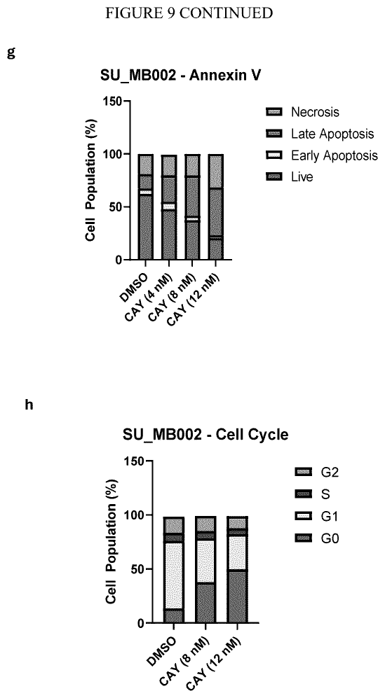

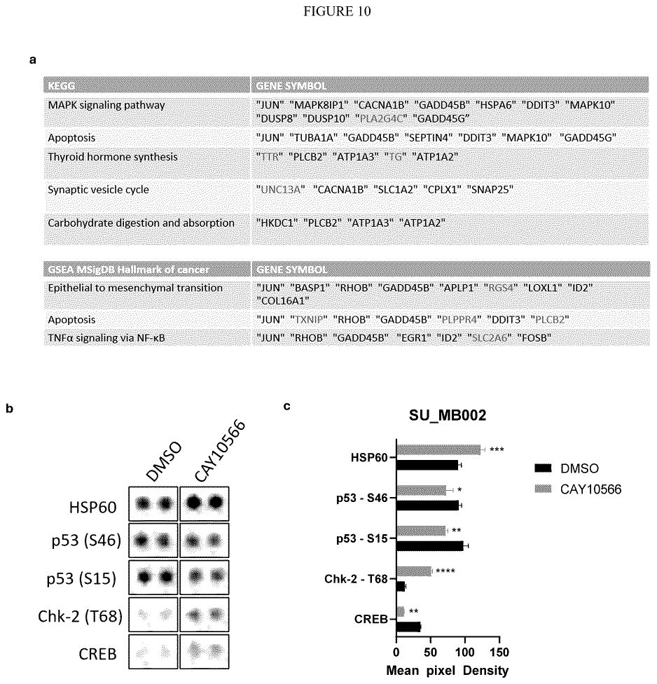

SCD1 inhibition triggers apoptosis and a cascade of oncogenic signaling programs. To unravel molecular mechanisms underlying SCD1 inhibition in G3-MB, the transcriptome of SU_MB002 cells was examined after 72 hr treatment with CAY10566 (IC50) via RNA-seq. Differential expression analysis on CAY10566 and DMSO control treated cells revealed 166 differentially expressed genes (DEGs) (adjusted p-value <0.05 and log 2FC>|1|). Of these DEGs, 138 were up-regulated and 28 were down-regulated (FIG. 9 a, b). Next, gene set enrichment analysis (GSEA) was performed. Interestingly, all DEGs were significantly enriched for bona fide cancer-related pathways including the MAPK signaling pathway, apoptosis, TNF-α signaling via NF-κB and epithelial-to-mesenchymal transition. Additionally, thyroid hormone synthesis, synaptic vesicle cycle and carbohydrate digestion and absorption pathways were detected (FIG. 9 c,d and FIG. 10 a). Interestingly thyroid hormone has been shown to suppress MB ⁴⁹. In addition to RNA-seq, kinase phosphorylation profiles were explored in SU_MB002 cells and compared differential expression levels between AAVS1 and SCD1 KO cells (FIG. 9 e,f), and between DMSO and CAY10566 treatment (FIG. 9 b,c). To this end, a phospho-kinase array kit (R&D Systems) was utilized, and it was found that the abundance of several phospho-kinase species was increased upon SCD1 KO (FIG. 9 e). Interestingly, significantly increased expression levels of phospho-proteins implicated in cell death mechanisms were observed including autophagy, ferroptosis and apoptosis (HSP60⁵⁰, STAT3^{51,52} p53⁵³, GSK-3α/β⁵⁴), lipid metabolism (STAT3⁵¹, RISK1/2/3⁵⁵, GSK-3α/β⁵⁴, p53⁵⁶) and metabolic pathways implicated in nutrient availability/cellular energetic status, mitochondrial functioning and metabolic reprogramming (HSP60⁵⁰, RSK1/2/3⁵⁵, p53-S3925⁵⁷, Akt-T308⁵⁸, GSK-3α/β^{54,59}, c-Jun⁶⁰), DNA damage (Chk2, p53)⁶¹, proliferation (HSP60⁶², Akt⁶³, GSK-3α/β⁵⁴), cancer stem cell self-renewal and treatment resistance (β-catenin⁶⁴, STAT3^{9,23}, Akt⁶³, GSK-3α/β⁵⁹)(FIG. 9 f). Importantly, increased expression of c-Jun-S63 was observed, which is likely a key player in the cellular response to SCD1 inhibition, resulting in pro-oncogenic and cell death evasion mechanisms. Moreover, these findings complement the RNA-seq DEG analysis (FIG. 9 f and FIG. 10). In addition, apoptosis was confirmed as the mechanism of cell death by subjecting SU_MB002 and HD-MB03 cells to multiple concentrations of CAY10566 and staining by Annexin V by FACs (FIG. 9 g). CAY10566 treatment induced cell cycle arrest, as determined by propidium iodide based FACS assessment of DNA content (FIG. 9 h).

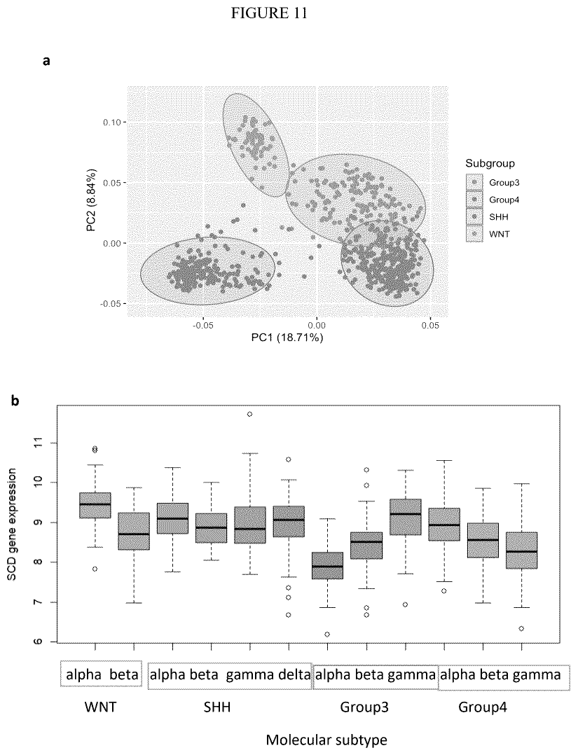

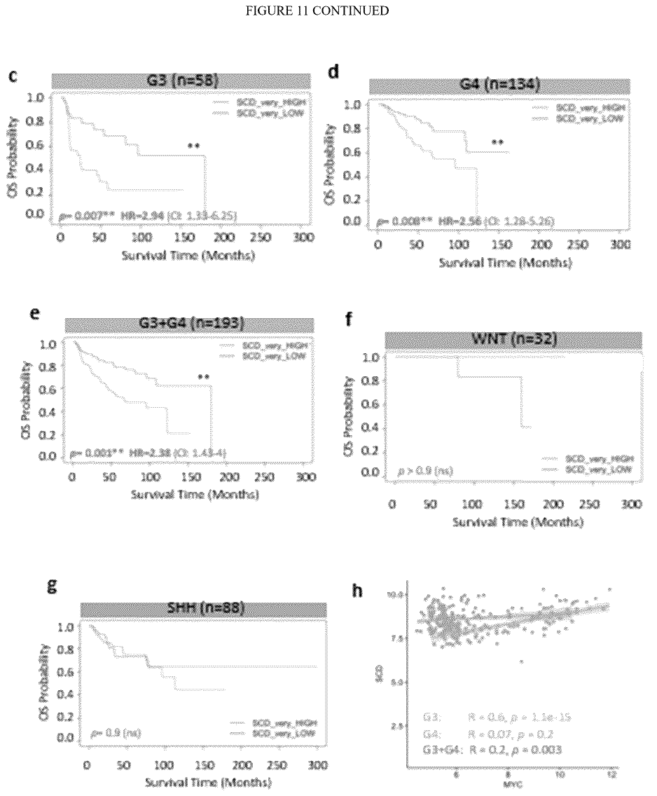

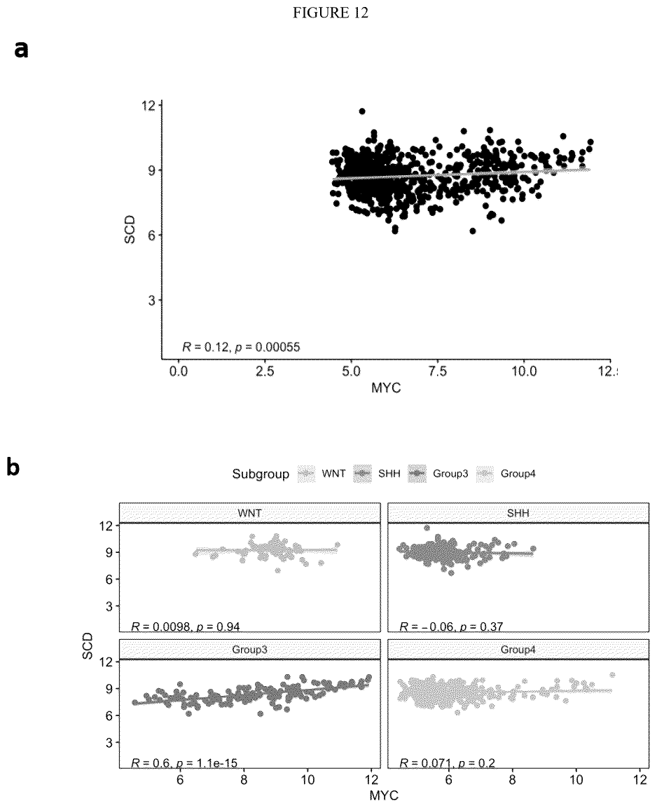

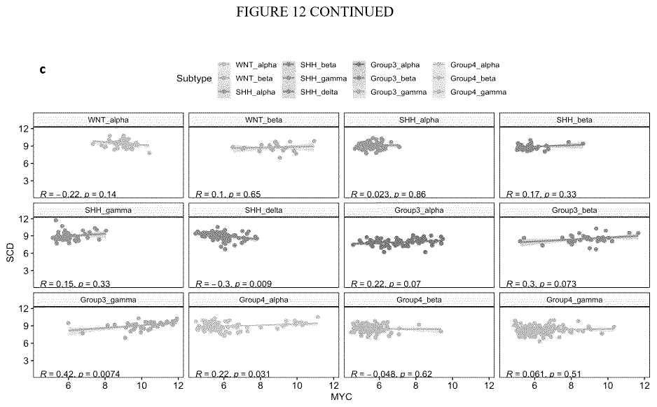

SCD gene expression is a prognostic marker in G3 and G4 MB patients. Having identified SCD1 as a therapeutic target in the in vitro and in vivo G3-MB models, the prognostic value of SCD expression was examined on a clinical level. To this end, the Cavalli microarray dataset containing tumor expression data from 763 MB patients (GSE85217)⁶⁵ was mined. Principal Component Analysis of normalized samples, colored by subgroup, revealed four clusters consistent with the MB subgroups (WNT, SHH, G3 and G4). An overlapping region of G3 and G4 can be observed (FIG. 11 a). This is consistent with a recent finding that G3 and G4 are not separate entities, but exist on a transcriptomic spectrum⁶⁶. Subsequently, SCD expression was investigated in relation to molecular subgroup and molecular subtype (FIG. 11 b-h and FIG. 12 a-c). Focusing on the molecular classification, significantly different expression levels were identified among subgroups and subtypes. The highest variability was observed within the G3 and G4 subtypes (alpha, beta, gamma) (FIG. 11 b). Considering the variability in G3 and G4, survival analysis separating both subgroups by very high SCD expression (upper quartile) and very low SCD expression (lower quartile) was performed. Interestingly, patients with very high SCD expression have a significantly reduced overall survival time in both G3 (p=0.007, HR=2.94, n=58) and G4 (p=0.008, HR=2.56, n=134) (FIG. 11 c, d), but not in the WNT nor SHH subgroups (FIG. 11 f, g). Since G3-MB and G4-MB vary the most in SCD expression, and these two subgroups have been shown to exist on a transcriptomic continuum, the G3-MB and G4-MB patient populations were pooled as one (G3+G4) and repeated the very high/very low survival analysis on this pooled population. Interestingly, a significantly reduced survival time is observed in high SCD expressing G3+G4 MB (p=0.001, HR=2.38, n=193 (FIG. 11 e), suggesting that SCD expression is a strong prognostic marker for patients in G3-MB and G4-MB subgroups.

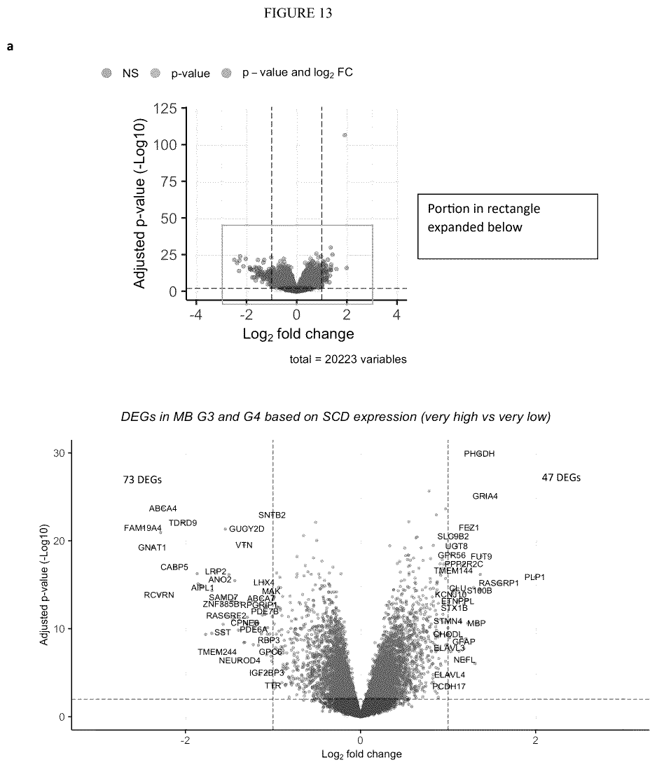

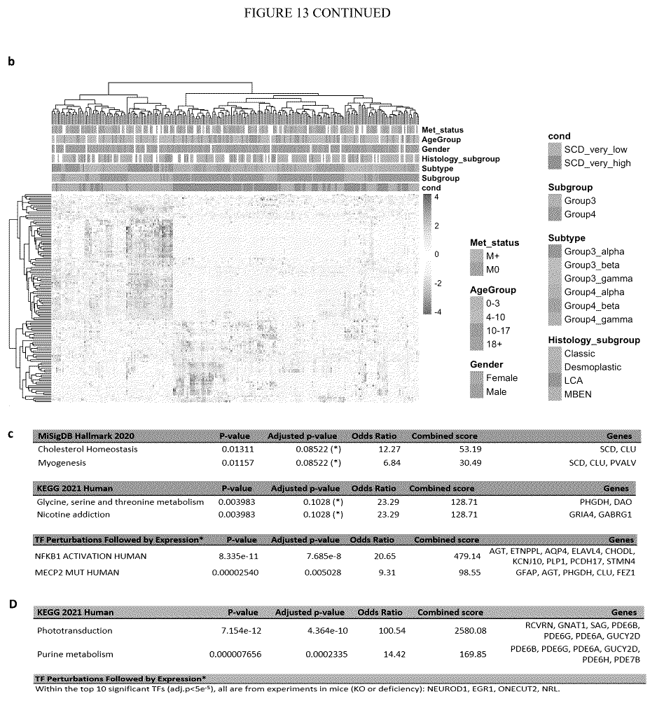

G3-MB and G4-MB patients may use distinct metabolic programs based on SCD expression: To further investigate molecular mechanisms that distinguish high SCD expressing patient tumors from low SCD expressing tumors, the Cavalli microarray dataset containing tumor expression data from 763 MB patients (GSE85217)⁶⁵ described above was mined and a differential expression analysis was performed on these two groups. The G3 and G4 subgroups were separated by very high SCD expression (upper quartile) and very low SCD expression (lower quartile).To ensure the detected mechanisms are SCD specific, and considering MYC is a potent oncogenic driver specific to G3-MB⁶⁵, it was first explored whether a correlation exists between SCD expression and MYC expression, and to determine whether MYC could be a confounding variable. A significant, medium-strong positive correlation was identified for G3-MB (R=0.6, p-value=1.1e-15) but not in G4-MB (R=0.07) nor in the pooled G3+G4 MB cohort (R=0.2) (FIG. 11 h and FIG. 12). Therefore, differential expression analysis was performed in the pooled G3+G4 MB patient subset for very high or very low SCD expression (n=236). The analysis revealed 120 differentially expressed genes (DEGs) (adjusted p-value <0.01 and log 2FC >|1|), consisting of 73 downregulated and 47 upregulated DEG (FIG. 13 a, b). Hierarchical clusters were observed for very low SCD expression, very high SCD expression, and intermediate groups. Within these groups, the proportion of G3-MB and G4-MB samples was not uniform, which may be explained by the G3/G4 transcriptomic spectrum (FIG. 13 b). Gene set enrichment analysis querying the KEGG and the GSEA MSigDB Hallmarks of Cancer databases revealed an enrichment in pathways including cholesterol homeostasis; myogenesis; glycine, serine, and threonine metabolism (with 10% FDR) for G3+G4 MB patients with very high SCD expression. In contrast, SCD very low patients may rely more on phototransduction and purine metabolism, as observed in the downregulation panel (adjusted p-value <0.001) (FIG. 13 c, d). Together, these findings indicate SCD as a potential prognostic marker and highlight underlying molecular mechanisms that can be explored as therapeutic targets.

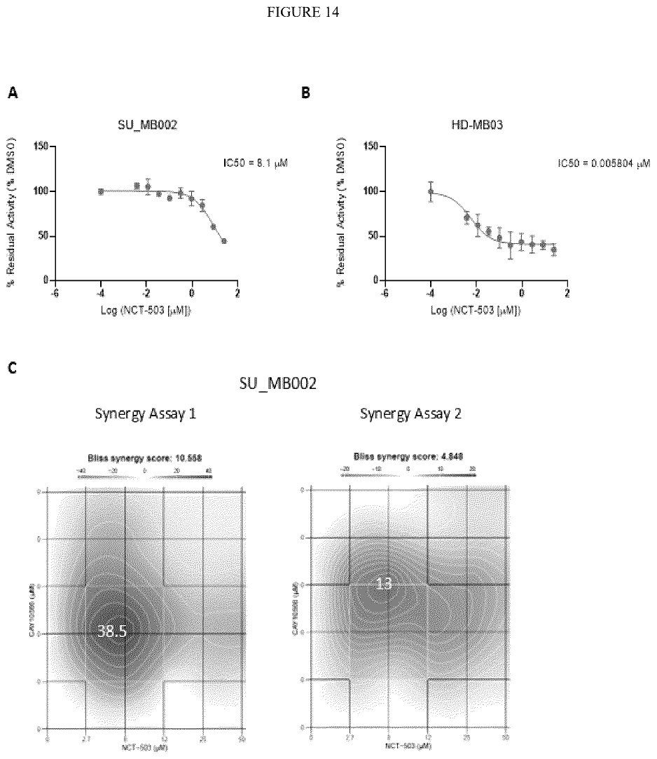

Further, one enzyme that clearly stood out as a DEG is phosphoglycerate dehydrogenase (PHGDH), the rate limiting enzyme in serine metabolism. Importantly, PHGDH is targetable by small molecules, such as NCT-503. Accordingly, NCT-503 inhibitor was tested on two MYC-amplified G3-MB lines (SU_MB002 and HD-MB03) and IC50 values were determined (FIG. 14 A and 14 B). A synergy assay was then performed on SU_MB002 cells with CAY10566 (SCD1 inhibitor) versus NCT-503 (PHGDH). A strong synergistic effect was found with the highest BLISS drug combination score reaching 38.5. We then repeated this assay finding similar results (FIG. 14 C).

### DISCUSSION

Activation of the De Novo Lipogenesis (DNL) pathway is gaining recognition as a hallmark of aggressive cancers^{18,38}. Indeed, fatty acid (FA) metabolism plays a central role in sustaining the highly proliferative tumor cell's increased lipid demands for energetics, structural cellular components, hypoxic stress resistance via oxidative phosphorylation and an improved redox balance¹². To accommodate these needs, cancer cells have developed a reliance on their fatty acid biosynthesis machinery for lipid production as well as lipotoxicity management. Stearoyl-CoA Desaturase 1 (SCD1) is a central component of DNL-mediated FA desaturation processes^{17,18,33}. SCD1 activity has been described in various cancers, including brain cancers such as GBM, and correlates with an aggressive phenotype, poor prognosis and treatment resistance²²⁻³¹.

As the rate-limiting enzyme in the FA desaturation cascade, SCD1 is central in balancing the opposing forces of high proliferation and lipid cytotoxicity in neoplasmic cells^{45,67} Pinkham et al, have demonstrated this role in GBM, where CSCs depend on SCD1 activation to maintain balanced levels of saturated FAs (sFA) and SCD1 inhibition leads to sFA accumulation and ER stress²⁴. Moreover, Pinkham et al, identified a resistance strategy through MEK/ERK and AMPK activation, resulting in mitigation of sFA toxicity by desaturating phospholipids and diverting toxic lipids into lipid droplets⁶⁸.

In this work, a differential abundance of lipid species and lipid saturation levels was observed between NSC and G3-MB cells, which presents a critical therapeutic opportunity in childhood medulloblastoma where sparing normal stem cell pools could mitigate the devastating neurodevelopmental and neurocognitive sequelae of current systemic therapies. The DNL pathway was identified as a therapeutic vulnerability in G3-MB which is upregulated after SOC, hereby presenting it as a key pathway underlying treatment resistance. It was found that SCD1 inhibition selectively targets G3-MB BTICs over normal NSCs with a potent 10,000-fold therapeutic window. Moreover, pharmacological and genetic targeting of SCD1 is both rescued by Oleic Acid (OA) supplementation, further indicating a prominent role of lipid homeostasis in G3-MB survival. Clonal KO cells of the essential SCD1 gene were generated by leveraging the protective properties of OA. In doing so, survival in the in vivo models was significantly improved. Importantly, translational utility was proven by demonstrating that G3-MB tumors are susceptible to in vivo small molecule treatment with the intracranial and flank models. SCD1 inhibition in G3-MB leads to a cascade of pro-oncogenic pathways regulating metabolic reprogramming, mitochondrial functioning, cancer stem-cell function and cell death evasion and ultimately results in cell cycle arrest and apoptosis. c-Jun, MAPK and NF-κB were identified as potential central players in these processes.

Combined, this work not only implicates the DNL pathway as a potent therapeutic target in MB, but also demonstrates a more fundamental concept; tumor cells struggle to manage the cytotoxic aftermath of lipid production during uncontrolled proliferation, rendering them reliant on low expressed genes, such as SCD1 in G3-MB to accommodate such toxicities. Furthermore, it was demonstrated that lipidomics and CRISPR technology present an excellent combination of data discovery platforms for uncovering such cancer-selective vulnerabilities.

Response to CAY10566 treatment in the intracranial PDX model was significant, although survival extension remained lower than expected. CAY10566 is known in literature as having a low blood-brain barrier penetrability. Indeed, Oatman et al, performed detailed pharmacokinetic studies with CAY10566 in mice and observed peak brain concentrations within 1 hour with a half-life of approximately 4 hours⁴⁸. The half-life of CAY10566 could be an indication the IP dosing regimen may not be adequate to reach full therapeutic potential within the brain. Badr et al, administered CAY10566 by intranasal delivery in their GBM model²⁷. This is a labor-intensive and delicate technique to master and unfortunately the efforts were not successful (data not shown). In their follow-up paper, the group used a BBB penetrant inhibitor of SCD1, YTX-773970⁶⁸. Unfortunately, it was not possible to obtain the compound from its manufacturer.

The treatment-induced pathways detected in the RNA-seq and phospho-kinase array are likely to induce treatment resistance and should be explored as potential targets for combinatorial treatment with SCD1 inhibitors. In proliferating cells, the upregulation of SCD1 expression by mitogenic factors induces the activation of pro-growth signaling cascades, including mitogen-induced kinases (MAPK, ERK1/2), JNK, and Akt⁶⁹⁻⁷¹. Additionally, SCD1 has been described to drive metastasis through dysregulated β-catenin function in a GSK3b-dependent manner⁷². Interestingly, JNK activation after SCD1 inhibition has also been described as an apoptotic switch after the inability to overcome SCD1 inhibition mediated ER stress²⁴. Our SOC-adapted murine model suggests an upregulation of the DNL pathway at relapse, which is in concordance with GBM models, where SCD1 upregulation promotes Temozolomide resistance via SCD1 TMZ resistance via Akt-GSK3β-β-Catenin axis⁵⁹.

Tumor cells can meet their metabolic demands by re-routing metabolites from different pathways. Targeting a metabolic pathway, like DNL, may prove ineffective due to such redundancies and treatment resistance may arise^{73,74}. For these reasons, the above-mentioned pathways should be explored as metabolic synthetically lethal combinations together with SCD1 treatment in MYC-amplified G3-MB. Additionally, future work should explore alternative inhibitors targeting the DNL enzymes, as the inhibitors used in this study may not be the most potent or effective⁷³.

Our analysis of the Cavalli dataset revealed SCD expression as a prognostic marker for poor survival in G3-MB and G4-MB. Comparison of very high and very low SCD expression patient tumor samples indicated that high SCD expressors may rely on lipid metabolism (cholesterol homeostasis) and amino-acid metabolism (glycine, serine, and threonine metabolism). In contrast, those with low SCD expression are enriched in nucleotide metabolism (purine metabolism).

These results point towards potential synergistic vulnerabilities for patients with high SCD expression. Inhibition of SCD1 in combination with statins may target one such vulnerability in HMG-CoA reductase, the rate-limiting enzyme in the mevalonate pathway, thereby blocking cholesterol production⁷⁵. Evidence suggest that statins (e.g. Simvastatin) have a risk-reducing effect on cancer, and their use as adjuvants in cancer treatment has been investigated in clinical trials⁷⁶. Anti-tumoral effects with cholesterol biosynthesis inhibitors have been demonstrated in SHH-MB in vivo models⁷⁷. These results are consistent with Park et al., that identified several genes from metabolic pathways, including the cholesterol (HMGCS1) and lipid synthesis (SCD, FASN) pathways, as potential prognostic markers for MB⁷⁸. PHGDH was identified as the most significantly upregulated gene, encoding the rate limiting enzyme of the serine biosynthetic pathway (3-phosphoglycerate dehydrogenase). Interestingly, PHGDH has been implicated as a resistance mechanism in metabolically driven cancers⁷⁹. Moreover, targeting glycine, serine and threonine metabolism may present other combinatory treatment strategies, as described in further detail below.

Of the wide variety of known SCD1 inhibitors, few have reached clinical trial. These clinical trials, mostly for Type 2 diabetes, fail due to adverse effects including eye dryness, hair loss, dryness of skin and subsequent cold-induced hypothermia, outweighing therapeutic effects. These side-effects, reported in the literature and observed in our animal models, are caused by the inhibition of SCD1-mediated production of MUFA products essential in eye lubricants and skin oils^{80,81}. A recent animal study has shown that mitigating strategies are possible as administration of a palmitoyl transferase inhibitor-based eye lubricant completely reversed adverse effects of eye lubricant dysfunction in SCD1 KO mice⁸². In addition, pro-drug strategies are being explored to overcome on-target off-site effects. Combining these mitigating and preventative measures strongly suggests that SCD1 is a clinically relevant target to pursue in aggressive cancers (including MB), where the therapeutic effects most likely will outweigh the side effects. The most pressing hurdle, however, is Blood-Brain-Barrier penetrability. Excitingly, YTX-7739, a BBB penetrable SCD1 inhibitor, has recently been demonstrated to have a potent therapeutic effect in GBM⁶⁸. Anticipating clinical trials with a BBB penetrable drug, SCD1 inhibition may interfere with axon myelination⁸³. Additionally, Oleic Acid is produced by neural stem cells and functions as a metabolic ligand that modulates neurogenesis in the brain⁸⁴. These potential toxicities should be considered for future clinical development.

Further, cholesterol homeostasis; myogenesis; glycine, serine, and threonine metabolism pathways were identified as regulated by SCD1 in G3 and G4 medulloblastoma. These pathways can yield potent targets for combinatorial treatment with SCD1 inhibitors, as demonstrated with the PHGDH inhibitor NCT-503.

Cancer-selective vulnerabilities are the new frontier for future treatment modalities. This work suggests that SCD expression is a prognostic marker that identifies patients within the most aggressive MB subgroups (G3 and G4). Selective targeting of G3-MB by SCD1 inhibition was demonstrated, while sparing normal cells. Moreover, SCD1 inhibition may act as a SOC de-escalator by impairing cancer cell membranes, thereby rendering them more susceptible to chemotherapies. Combined, this improved treatment modality has the potential of reducing life-long neurocognitive and neurodevelopmental deficits, which remains an urgent unmet need for pediatric G3 and G4 MB patients.

### EMBODIMENTS OF THE APPLICATION

1. A method of treating high-risk medulloblastoma comprising administering a therapeutically effective amount of a stearoyl-CoA Desaturase 1 (SCD1) inhibitor to a subject in need thereof.

2. The method of embodiment 1, wherein treating high-risk medulloblastoma further comprises administering a therapeutically effective amount of an SCD1 inhibitor in combination with another known agent useful for treating a high-risk medulloblastoma.

3. The method of embodiment 1, wherein treating high-risk medulloblastoma further comprises administering a therapeutically effective amount of an SCD1 inhibitor in combination with an agent useful for inhibiting de novo lipid synthesis.

4. The method of embodiment 1 wherein treating high-risk medulloblastoma further comprises administering a therapeutically effective amount of an SCD1 inhibitor in combination with an agent useful for inhibiting pro-growth signaling cascades, including mitogen-induced kinases (MAPK, ERK1/2), JNK, and Akt, or β-catenin, GSK3b, JNK.

5. The method of embodiment 2, wherein the known agent useful for treating a high-risk medulloblastoma comprises surgical resection, chemotherapy or cranio-spinal irradiation.

6. The method of embodiment 1, wherein the high risk medulloblastoma comprises recurrent medulloblastoma.

7. The method of embodiment 1, wherein the high risk medulloblastoma comprises MYC-driven Group 3 MB (G3-MB) or MYC-driven Group 4 MB (G4-MB) in a subject in need thereof.

8. The method of embodiment 1, comprising administering the SCD1 inhibitor by intraperitoneal (IP) injections, intranasally, intracranially, intravenously, or orally.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### Full Citations for Documents Referred to in the Specification

A number of publications are cited herein. Full citations for these references are provided below. Each of these references is incorporated herein by reference in its entirety into the present disclosure, to the same extent as if each individual reference was specifically and individually indicated to be incorporated by reference.
(1) Ostrom, Q. T.; Price, M.; Neff, C.; Cioffi, G.; Waite, K. A.; Kruchko, C.; Barnholtz-Sloan, J. S. CBTRUS Statistical Report: Primary Brain and Other Central Nervous System Tumors Diagnosed in the United States in 2015-2019. Neuro-Oncology 2022, 24 (Supplement_5), v1-v95.
(2) Hill, R. M.; Richardson, S.; Schwalbe, E. C.; Hicks, D.; Lindsey, J. C.; Crosier, S.; Rafiee, G.; Grabovska, Y.; Wharton, S. B.; Jacques, T. S.; Michalski, A.; Joshi, A.; Pizer, B.; Williamson, D.; Bailey, S.; Clifford, S. C. Time, Pattern, and Outcome of Medulloblastoma Relapse and Their Association with Tumour Biology at Diagnosis and Therapy: A Multicentre Cohort Study. Lancet Child Adolesc Health 2020, 4 (12), 865-874.
(3) Sabel, M.; Fleischhack, G.; Tippelt, S.; Gustafsson, G.; Doz, F.; Kortmann, R.; Massimino, M.; Navajas, A.; von Hoff, K.; Rutkowski, S.; Warmuth-Metz, M.; Clifford, S. C.; Pietsch, T.; Pizer, B.; Lannering, B.; SIOP-E Brain Tumour Group. Relapse Patterns and Outcome after Relapse in Standard Risk Medulloblastoma: A Report from the HIT-SIOP-PNET4 Study. J Neurooncol 2016, 129 (3), 515-524.
(4) Singh, S. K.; Clarke, I. D.; Terasaki, M.; Bonn, V. E.; Hawkins, C.; Squire, J.; Dirks, P. B. Identification of a Cancer Stem Cell in Human Brain Tumors. Cancer Research 2003, 63 (18), 5821-5828.
(5) Singh, S. K.; Hawkins, C.; Clarke, I. D.; Squire, J. A.; Bayani, J.; Hide, T.; Henkelman, R. M.; Cusimano, M. D.; Dirks, P. B. Identification of Human Brain Tumour Initiating Cells. Nature 2004, 432 (7015), 396-401.
(6) Bao, S.; Wu, Q.; McLendon, R. E.; Hao, Y.; Shi, Q.; Hjelmeland, A. B.; Dewhirst, M. W.; Bigner, D. D.; Rich, J. N. Glioma Stem Cells Promote Radioresistance by Preferential Activation of the DNA Damage Response. Nature 2006, 444 (7120), 756-760.
(7) Nakai, E.; Park, K.; Yawata, T.; Chihara, T.; Kumazawa, A.; Nakabayashi, H.; Shimizu, K. Enhanced MDR1 Expression and Chemoresistance of Cancer Stem Cells Derived from Glioblastoma. Cancer Investigation 2009, 27 (9), 901-908.
(8) Ellison, D. W.; Clifford, S. C.; Gajjar, A.; Gilbertson, R. J. What's New in Neuro-Oncology?Recent Advances in Medulloblastoma. Eur J Paediatr Neurol 2003, 7 (2), 53-66.
(9) Bakhshinyan, D.; Adile, A. A.; Liu, J.; Gwynne, W. D.; Suk, Y.; Custers, S.; Burns, I.; Singh, M.; McFarlane, N.; Subapanditha, M. K.; Qazi, M. A.; Vora, P.; Kameda-Smith, M. M.; Savage, N.; Desmond, K. L.; Tatari, N.; Tran, D.; Seyfrid, M.; Hope, K.; Bock, N. A.; Venugopal, C.; Bader, G. D.; Singh, S. K. Temporal Profiling of Therapy Resistance in Human Medulloblastoma Identifies Novel Targetable Drivers of Recurrence. Sci Adv 2021, 7 (50), eabi5568.
(10) Hanahan, D. Hallmarks of Cancer: New Dimensions. Cancer Discovery 2022, 12 (1), 31-46.
(11) Zhu, X.; Chen, H.-H.; Gao, C.-Y.; Zhang, X.-X.; Jiang, J.-X.; Zhang, Y.; Fang, J.; Zhao, F.; Chen, Z.-G. Energy Metabolism in Cancer Stem Cells. World J Stem Cells 2020, 12 (6), 448-461.
(12) Menendez, J. A.; Lupu, R. Fatty Acid Synthase-Catalyzed de Novo Fatty Acid Biosynthesis: From Anabolic-Energy-Storage Pathway in Normal Tissues to Jack-of-All-Trades in Cancer Cells. Arch Immunol Ther Exp (Warsz) 2004, 52 (6), 414-426.
(13) Menendez, J. A.; Lupu, R. Fatty Acid Synthase and the Lipogenic Phenotype in Cancer Pathogenesis. Nat Rev Cancer 2007, 7 (10), 763-777.
(14) Currie, E.; Schulze, A.; Zechner, R.; Walther, T. C.; Farese, R. V. Cellular Fatty Acid Metabolism and Cancer. Cell Metab 2013, 18 (2), 153-161.
(15) Swinnen, J. V.; Brusselmans, K.; Verhoeven, G. Increased Lipogenesis in Cancer Cells: New Players, Novel Targets. Curr Opin Clin Nutr Metab Care 2006, 9 (4), 358-365.
(16) Zaidi, N.; Swinnen, J. V.; Smans, K. ATP-Citrate Lyase: A Key Player in Cancer Metabolism. Cancer Res 2012, 72 (15), 3709-3714.
(17) Guertin, D. A.; Wellen, K. E. Acetyl-CoA Metabolism in Cancer. Nat Rev Cancer 2023, 23 (3), 156-172.
(18) Munir, R.; Lisec, J.; Swinnen, J. V.; Zaidi, N. Too Complex to Fail?Targeting Fatty Acid Metabolism for Cancer Therapy. Progress in Lipid Research 2022, 85, 101143.
(19) Scott, K. E. N.; Wheeler, F. B.; Davis, A. L.; Thomas, M. J.; Ntambi, J. M.; Seals, D. F.; Kridel, S. J. Metabolic Regulation of Invadopodia and Invasion by Acetyl-CoA Carboxylase 1 and de Novo Lipogenesis. PLoS One 2012, 7 (1), e29761.
(20) Hodson, L.; Fielding, B. A. Stearoyl-CoA Desaturase: Rogue or Innocent Bystander?Prog Lipid Res 2013, 52 (1), 15-42.
(21) Paton, C. M.; Ntambi, J. M. Biochemical and Physiological Function of Stearoyl-CoA Desaturase. Am J Physiol Endocrinol Metab 2009, 297 (1), E28-37.
(22) Zhao, G.; Tan, Y.; Cardenas, H.; Vayngart, D.; Huang, H.; Wang, Y.; Keathley, R.; Wei, J.-J.; Ferreira, C. R.; Cheng, J.-X.; Matei, D. The Balance between Saturated and Unsaturated Fatty Acids Regulates Ovarian Cancer Cell Fate. bioRxiv May 25, 2022, p 2022.05.24.493247.
(23) Vivas-Garcia, Y.; Falletta, P.; Liebing, J.; Louphrasitthiphol, P.; Feng, Y.; Chauhan, J.; Scott, D. A.; Glodde, N.; Chocarro-Calvo, A.; Bonham, S.; Osterman, A. L.; Fischer, R.; Ronai, Z.; Garcia-Jimenez, C.; H6lzel, M.; Goding, C. R. Lineage-Restricted Regulation of SCD and Fatty Acid Saturation by MITF Controls Melanoma Phenotypic Plasticity. Mol Cell 2020, 77 (1), 120-137.e9.
(24) Pinkham, K.; Park, D. J.; Hashemiaghdam, A.; Kirov, A. B.; Adam, I.; Rosiak, K.; da Hora, C. C.; Teng, J.; Cheah, P. S.; Carvalho, L.; Ganguli-Indra, G.; Kelly, A.; Indra, A. K.; Badr, C. E. Stearoyl CoA Desaturase Is Essential for Regulation of Endoplasmic Reticulum Homeostasis and Tumor Growth in Glioblastoma Cancer Stem Cells. Stem Cell Reports 2019, 12 (4), 712-727.
Zhang, J.; Song, F.; Zhao, X.; Jiang, H.; Wu, X.; Wang, B.; Zhou, M.; Tian, M.; Shi, B.; Wang, H.; Jia, Y.; Wang, H.; Pan, X.; Li, Z. EGFR Modulates Monounsaturated Fatty Acid Synthesis through Phosphorylation of SCD1 in Lung Cancer. Molecular Cancer 2017, 16 (1), 127.
(26) Piccinin, E.; Cariello, M.; Moschetta, A. Lipid Metabolism in Colon Cancer: Role of Liver X Receptor (LXR) and Stearoyl-CoA Desaturase 1 (SCD1). Molecular Aspects of Medicine 2021, 78, 100933.
(27) Wang, C.; Shi, M.; Ji, J.; Cai, Q.; Zhao, Q.; Jiang, J.; liu, J.; Zhang, H.; Zhu, Z.; Zhang, J. Stearoyl-CoA Desaturase 1 (SCD1) Facilitates the Growth and Anti-Ferroptosis of Gastric Cancer Cells and Predicts Poor Prognosis of Gastric Cancer. Aging (Albany NY) 2020, 12 (15), 15374-15391.
(28) Yahagi, N.; Shimano, H.; Hasegawa, K.; Ohashi, K.; Matsuzaka, T.; Najima, Y.; Sekiya, M.; Tomita, S.; Okazaki, H.; Tamura, Y.; lizuka, Y.; Ohashi, K.; Nagai, R.; Ishibashi, S.; Kadowaki, T.; Makuuchi, M.; Ohnishi, S.; Osuga, J.; Yamada, N. Co-Ordinate Activation of Lipogenic Enzymes in Hepatocellular Carcinoma. Eur J Cancer 2005, 41 (9), 1316-1322. 7.
(29) Wang, J.; Xu, Y.; Zhu, L.; Zou, Y.; Kong, W.; Dong, B.; Huang, J.; Chen, Y.; Xue, W.; Huang, Y.; Zhang, J. High Expression of Stearoyl-CoA Desaturase 1 Predicts Poor Prognosis in Patients with Clear-Cell Renal Cell Carcinoma. PLOS ONE 2016, 11 (11), e0166231.
(30) Holder, A. M.; Gonzalez-Angulo, A. M.; Chen, H.; Akcakanat, A.; Do, K.-A.; Symmans, W. F.; Pusztai, L.; Hortobagyi, G. N.; Mills, G. B.; Meric-Bernstam, F. High Stearoyl-CoA Desaturase 1 Expression Is Associated with Shorter Survival in Breast Cancer Patients. Breast Cancer Res Treat 2013, 137 (1), 319-327.
(31) Presler, M.; Wojtczyk-Miaskowska, A.; Schlichtholz, B.; Kaluzny, A.; Matuszewski, M.; Mika, A.; Sledzinski, T.; Swierczynski, J. Increased Expression of the Gene Encoding Stearoyl-CoA Desaturase 1 in Human Bladder Cancer. Mol Cell Biochem 2018, 447 (1), 217-224.
(32) Liu, G.; Feng, S.; Jia, L.; Wang, C.; Fu, Y.; Luo, Y. Lung Fibroblasts Promote Metastatic Colonization through Upregulation of Stearoyl-CoA Desaturase 1 in Tumor Cells. Oncogene 2018, 37 (11), 1519-1533.
(33) Budhu, A.; Roessler, S.; Zhao, X.; Yu, Z.; Forgues, M.; Ji, J.; Karoly, E.; Qin, L.-X.; Ye, Q.-H.; Jia, H.-L.; Fan, J.; Sun, H.-C.; Tang, Z.-Y.; Wang, X. W. Integrated Metabolite and Gene Expression Profiles Identify Lipid Biomarkers Associated with Progression of Hepatocellular Carcinoma and Patient Outcomes. Gastroenterology 2013, 144 (5), 1066-1075.e1.
(34) Peck, B.; Schulze, A. Lipid Desaturation—the next Step in Targeting Lipogenesis in Cancer?FEBS J 2016, 283 (15), 2767-2778.
(35) von Roemeling, C. A.; Marlow, L. A.; Pinkerton, A. B.; Crist, A.; Miller, J.; Tun, H. W.; Smallridge, R. C.; Copland, J. A. Aberrant Lipid Metabolism in Anaplastic Thyroid Carcinoma Reveals Stearoyl CoA Desaturase 1 as a Novel Therapeutic Target. J Clin Endocrinol Metab 2015, 100 (5), E697-709.
(36) Bansal, S.; Berk, M.; Alkhouri, N.; Partrick, D. A.; Fung, J. J.; Feldstein, A. Stearoyl-CoA Desaturase Plays an Important Role in Proliferation and Chemoresistance in Human Hepatocellular Carcinoma. J Surg Res 2014, 186 (1), 29-38.
(37) Roongta, U. V.; Pabalan, J. G.; Wang, X.; Ryseck, R.-P.; Fargnoli, J.; Henley, B. J.; Yang, W.-P.; Zhu, J.; Madireddi, M. T.; Lawrence, R. M.; Wong, T. W.; Rupnow, B. A. Cancer Cell Dependence on Unsaturated Fatty Acids Implicates Stearoyl-CoA Desaturase as a Target for Cancer Therapy. Molecular Cancer Research 2011, 9 (11), 1551-1561.
(38) Rysman, E.; Brusselmans, K.; Scheys, K.; Timmermans, L.; Derua, R.; Munck, S.; Van Veldhoven, P. P.; Waltregny, D.; Daniels, V. W.; Machiels, J.; Vanderhoydonc, F.; Smans, K.; Waelkens, E.; Verhoeven, G.; Swinnen, J. V. De Novo Lipogenesis Protects Cancer Cells from Free Radicals and Chemotherapeutics by Promoting Membrane Lipid Saturation. Cancer Res 2010, 70 (20), 8117-8126.
(39) Iwabuchi, K.; Nakayama, H.; Iwahara, C.; Takamori, K. Significance of Glycosphingolipid Fatty Acid Chain Length on Membrane Microdomain-Mediated Signal Transduction. FEBS Lett 2010, 584 (9), 1642-1652.
(40) Simons, K.; Toomre, D. Lipid Rafts and Signal Transduction. Nat Rev Mol Cell Biol 2000, 1 (1), 31-39.
(41) Broadfield, L. A.; Pane, A. A.; Talebi, A.; Swinnen, J. V.; Fendt, S.-M. Lipid Metabolism in Cancer: New Perspectives and Emerging Mechanisms. Dev Cell 2021, 56 (10), 1363-1393.
(42) Hoy, A. J.; Nagarajan, S. R.; Butler, L. M. Tumour Fatty Acid Metabolism in the Context of Therapy Resistance and Obesity. Nat Rev Cancer 2021, 21 (12), 753-766.
(43) Gwynne, W. D.; Suk, Y.; Custers, S.; Mikolajewicz, N.; Chan, J. K.; Zador, Z.; Chafe, S. C.; Zhai, K.; Escudero, L.; Zhang, C.; Zaslaver, O.; Chokshi, C.; Shaikh, M. V.; Bakhshinyan, D.; Burns, I.; Chaudhry, I.; Nachmani, O.; Mobilio, D.; Maich, W. T.; Mero, P.; Brown, K. R.; Quaile, A. T.; Venugopal, C.; Moffat, J.; Montenegro-Burke, J. R.; Singh, S. K. Cancer-Selective Metabolic Vulnerabilities in MYC-Amplified Medulloblastoma. Cancer Cell 2022, 40 (12), 1488-1502.e7.
(44) Vladoiu, M. C.; et al, Childhood Cerebellar Tumours Mirror Conserved Fetal Transcriptional Programs. Nature 2019, 572 (7767), 67-73.
(45) Nolan, C. J.; Larter, C. Z. Lipotoxicity: Why Do Saturated Fatty Acids Cause and Monounsaturates Protect against It?J Gastroenterol Hepatol 2009, 24 (5), 703-706.
(46) Gouw, A. M.; Margulis, K.; Liu, N. S.; Raman, S. J.; Mancuso, A.; Toal, G. G.; Tong, L.; Mosley, A.; Hsieh, A. L.; Sullivan, D. K.; Stine, Z. E.; Altman, B. J.; Schulze, A.; Dang, C. V.; Zare, R. N.; Felsher, D. W. The MYC Oncogene Cooperates with Sterol-Regulated Element-Binding Protein to Regulate Lipogenesis Essential for Neoplastic Growth. Cell Metab 2019, 30 (3), 556-572.e5.
(47) Toledo, C. M.; et al., Genome-Wide CRISPR-Cas9 Screens Reveal Loss of Redundancy between PKMYT1 and WEE1 in Glioblastoma Stem-like Cells. Cell Rep 2015, 13 (11), 2425-2439.
(48) Oatman, N.; Dasgupta, N.; Arora, P.; Choi, K.; Gawali, M. V.; Gupta, N.; Parameswaran, S.; Salomone, J.; Reisz, J. A.; Lawler, S.; Furnari, F.; Brennan, C.; Wu, J.; Sallans, L.; Gudelsky, G.; Desai, P.; Gebelein, B.; Weirauch, M. T.; D'Alessandro, A.; Komurov, K.; Dasgupta, B. Mechanisms of Stearoyl CoA Desaturase Inhibitor Sensitivity and Acquired Resistance in Cancer. Science Advances 2021, 7 (7), eabd7459.
(49) Yang, Y.; Valdes-Rives, S. A.; Liu, Q.; Gao, T.; Burudpakdee, C.; Li, Y.; Tan, J.; Tan, Y.; Koch, C. A.; Rong, Y.; Houser, S. R.; Wei, S.; Cai, K. Q.; Wu, J.; Cheng, S.; Wechsler-Reya, R.; Yang, Z. Thyroid Hormone Suppresses Medulloblastoma Progression through Promoting Terminal Differentiation of Tumor Cells. Cancer Cell 2024, 42 (8), 1434-1449.e5.
(50) Zhou, C.; Sun, H.; Zheng, C.; Gao, J.; Fu, Q.; Hu, N.; Shao, X.; Zhou, Y.; Xiong, J.; Nie, K.; Zhou, H.; Shen, L.; Fang, H.; Lyu, J. Oncogenic HSP60 Regulates Mitochondrial Oxidative Phosphorylation to Support Erk1/2 Activation during Pancreatic Cancer Cell Growth. Cell Death Dis 2018, 9 (2), 1-14.
(51) Tse, C.; Warner, A.; Farook, R.; Cronin, J. G. Phytochemical Targeting of STAT3 Orchestrated Lipid Metabolism in Therapy-Resistant Cancers. Biomolecules 2020, 10 (8), 1118.
(52) Ouyang, S.; Li, H.; Lou, L.; Huang, Q.; Zhang, Z.; Mo, J.; Li, M.; Lu, J.; Zhu, K.; Chu, Y.; Ding, W.; Zhu, J.; Lin, Z.; Zhong, L.; Wang, J.; Yue, P.; Turkson, J.; Liu, P.; Wang, Y.; Zhang, X. Inhibition of STAT3-Ferroptosis Negative Regulatory Axis Suppresses Tumor Growth and Alleviates Chemoresistance in Gastric Cancer. Redox Biology 2022, 52, 102317.
(53) Jin, H.-R.; Wang, J.; Wang, Z.-J.; Xi, M.-J.; Xia, B.-H.; Deng, K.; Yang, J.-L. Lipid Metabolic Reprogramming in Tumor Microenvironment: From Mechanisms to Therapeutics. Journal of Hematology & Oncology 2023, 16 (1), 103.
(54) Papadopoli, D.; Pollak, M.; Topisirovic, I. The Role of GSK3 in Metabolic Pathway Perturbations in Cancer. Biochimica et Biophysica Acta (BBA)-Molecular Cell Research 2021, 1868 (8), 119059.
(55) Caron, A.; Richard, D.; Laplante, M. The Roles of mTOR Complexes in Lipid Metabolism. Annual Review ofNutrition 2015, 35 (1), 321-348.
(56) Chen, L.-L.; Wang, W.-J. P53 Regulates Lipid Metabolism in Cancer. Int J Biol Macromol 2021, 192, 45-54.
(57) Castrogiovanni, C.; Waterschoot, B.; De Backer, O.; Dumont, P. Serine 392 Phosphorylation Modulates P53 Mitochondrial Translocation and Transcription-Independent Apoptosis. Cell Death Differ 2018, 25 (1), 190-203.
(58) Tsai, P.-J.; Lai, Y.-H.; Manne, R. K.; Tsai, Y.-S.; Sarbassov, D.; Lin, H.-K. Akt: A Key Transducer in Cancer. Journal of Biomedical Science 2022, 29 (1), 76.
(59) Dai, S.; Yan, Y.; Xu, Z.; Zeng, S.; Qian, L.; Huo, L.; Li, X.; Sun, L.; Gong, Z. SCD1 Confers Temozolomide Resistance to Human Glioma Cells via the Akt/GSK3p/p-Catenin Signaling Axis. Frontiers in Pharmacology 2018, 8.
(60) Serna, R.; Ramrakhiani, A.; Hernandez, J. C.; Chen, C.-L.; Nakagawa, C.; Machida, T.; Ray, R. B.; Zhan, X.; Tahara, S. M.; Machida, K. C-JUN Inhibits mTORC2 and Glucose Uptake to Promote Self-Renewal and Obesity. iScience 2022, 25 (6), 104325.
(61) Ward, I. M.; Wu, X.; Chen, J. Threonine 68 of Chk2 Is Phosphorylated at Sites of DNA Strand Breaks. J Biol Chem 2001, 276 (51), 47755-47758.
(62) Tang, H.; Li, J.; Liu, X.; Wang, G.; Luo, M.; Deng, H. Down-Regulation of HSP60 Suppresses the Proliferation of Glioblastoma Cells via the ROS/AMPK/mTOR Pathway. Sci Rep 2016, 6 (1), 28388.
(63) Cai, Q.; Zhou, W.; Wang, W.; Dong, B.; Han, D.; Shen, T.; Creighton, C. J.; Moore, D. D.; Yang, F. MAPK6-AKT Signaling Promotes Tumor Growth and Resistance to mTOR Kinase Blockade. Science Advances 2021, 7 (46), eabi6439.
(64) de Sousa e Melo, F.; Vermeulen, L. Wnt Signaling in Cancer Stem Cell Biology. Cancers (Basel) 2016, 8 (7), 60.
(65) Cavalli, F. M. G.; et al., Intertumoral Heterogeneity within Medulloblastoma Subgroups. Cancer Cell 2017, 31 (6), 737-754.e6.
(66) Williamson, D.; Schwalbe, E. C.; Hicks, D.; Aldinger, K. A.; Lindsey, J. C.; Crosier, S.; Richardson, S.; Goddard, J.; Hill, R. M.; Castle, J.; Grabovska, Y.; Hacking, J.; Pizer, B.; Wharton, S. B.; Jacques, T. S.; Joshi, A.; Bailey, S.; Clifford, S. C. Medulloblastoma Group 3 and 4 Tumors Comprise a Clinically and Biologically Significant Expression Continuum Reflecting Human Cerebellar Development. Cell Rep 2022, 40 (5), 111162.
(67) Scaglia, N.; Igal, R. A. Stearoyl-CoA Desaturase Is Involved in the Control of Proliferation, Anchorage-Independent Growth, and Survival in Human Transformed Cells. J Biol Chem 2005, 280 (27), 25339-25349.
(68) Eyme, K. M.; Sammarco, A.; Jha, R.; Mnatsakanyan, H.; Neustadt, R.; Moses, C.; Alnasser, A.; Tardiff, D.; Su, B.; Williams, K. J.; Bensinger, S. J.; Chung, C. Y.; Badr, C. E. Divergent MEK/ERK and AMPK Signaling Dictate Lipogenic Plasticity and Dependence on Fatty Acid Synthesis in Glioblastoma. bioRxiv Apr. 8, 2022, p 2022.04.07.487530.
(69) Igal, R. A. Roles of StearoylCoA Desaturase-1 in the Regulation of Cancer Cell Growth, Survival and Tumorigenesis. Cancers (Basel) 2011, 3 (2), 2462-2477.
(70) Mauvoisin, D.; Mounier, C. Hormonal and Nutritional Regulation of SCD1 Gene Expression. Biochimie 2011, 93 (1), 78-86.
(71) Porstmann, T.; Santos, C. R.; Griffiths, B.; Cully, M.; Wu, M.; Leevers, S.; Griffiths, J. R.; Chung, Y.-L.; Schulze, A. SREBP Activity Is Regulated by mTORC1 and Contributes to Akt-Dependent Cell Growth. Cell Metab 2008, 8 (3), 224-236.
(72) Wheelock, M. J.; Johnson, K. R. Cadherins as Modulators of Cellular Phenotype. Annu Rev Cell Dev Biol 2003, 19, 207-235.
(73) Munir, R.; Lisec, J.; Swinnen, J. V.; Zaidi, N. Too Complex to Fail?Targeting Fatty Acid Metabolism for Cancer Therapy. Progress in Lipid Research 2022, 85, 101143.
(74) Aregger, M.; Lawson, K. A.; Billmann, M.; Costanzo, M.; Tong, A. H. Y.; Chan, K.; Rahman, M.; Brown, K. R.; Ross, C.; Usaj, M.; Nedyalkova, L.; Sizova, O.; Habsid, A.; Pawling, J.; Lin, Z.-Y.; Abdouni, H.; Wong, C. J.; Weiss, A.; Mero, P.; Dennis, J. W.; Gingras, A.-C.; Myers, C. L.; Andrews, B. J.; Boone, C.; Moffat, J. Systematic Mapping of Genetic Interactions for de Novo Fatty Acid Synthesis Identifies C12orf49 as a Regulator of Lipid Metabolism. Nat Metab 2020, 2 (6), 499-513.
(75) Kofink, D.; Eppinga, R. N.; van Gilst, W. H.; Bakker, S. J. L.; Dullaart, R. P. F.; van der Harst, P.; Asselbergs, F. W. Statin Effects on Metabolic Profiles: Data From the PREVEND IT (Prevention of Renal and Vascular End-Stage Disease Intervention Trial). Circ Cardiovasc Genet 2017, 10 (6), e001759.
(76) Barbalata, C. I.; Tefas, L. R.; Achim, M.; Tomuta, I.; Porfire, A. S. Statins in Risk-Reduction and Treatment of Cancer. World J Clin Oncol 2020, 11 (8), 573-588.
(77) Fan, Q.; Gong, T.; Zheng, C.; Ng, J. M. Y.; Chen, J.; Myers, C.; Hensley, H.; Curran, T.; Yang, Z. Statins Repress Hedgehog Signaling in Medulloblastoma with No Bone Toxicities. Oncogene 2021, 40 (12), 2258-2272.
(78) Park, A. K.; Lee, J. Y.; Cheong, H.; Ramaswamy, V.; Park, S.-H.; Kool, M.; Phi, J. H.; Choi, S. A.; Cavalli, F.; Taylor, M. D.; Kim, S.-K. Subgroup-Specific Prognostic Signaling and Metabolic Pathways in Pediatric Medulloblastoma. BMC Cancer 2019, 19 (1), 571.
(79) Rathore, R.; Schutt, C. R.; Van Tine, B. A. PHGDH as a Mechanism for Resistance in Metabolically-Driven Cancers. Cancer Drug Resist 2020, 3 (4), 762-774.
(80) Tracz-Gaszewska, Z.; Dobrzyn, P. Stearoyl-CoA Desaturase 1 as a Therapeutic Target for the Treatment of Cancer. Cancers (Basel) 2019, 11 (7), 948.
(81) Ascenzi, F.; De Vitis, C.; Maugeri-Sacca, M.; Napoli, C.; Ciliberto, G.; Mancini, R. SCD1, Autophagy and Cancer: Implications for Therapy. Journal of Experimental & Clinical Cancer Research 2021, 40 (1), 265.
(82) Ji, C.; Guo, Y.; Liu, Y.; Xu, S.; Zhao, S.; Luo, X.; Qiu, F.; Huang, R.; Xu, Q.; Zheng, R.; Xia, M.; Zhao, Y.; Ren, J.; Qiu, Y. Inhibition of Ceramide de Novo Synthesis Ameliorates Meibomian Gland Dysfunction Induced by SCD1 Deficiency. The Ocular Surface 2021, 22, 230-241.
(83) Lorico, A.; Rappa, G.; Corbeil, D.; Le, T. T. Observation-Driven Inquiry: Raman Spectroscopic Imaging Illuminates Cancer Lipid Metabolism. Stem Cell Investig 2017, 4, 42.
(84) Kandel, P.; Semerci, F.; Mishra, R.; Choi, W.; Bajic, A.; Baluya, D.; Ma, L.; Chen, K.; Cao, A. C.; Phongmekhin, T.; Matinyan, N.; Jimenez-Panizo, A.; Chamakuri, S.; Raji, I. O.; Chang, L.; Fuentes-Prior, P.; MacKenzie, K. R.; Benn, C. L.; Estebanez-Perpina, E.; Venken, K.; Moore, D. D.; Young, D. W.; Maletic-Savatic, M. Oleic Acid Is an Endogenous Ligand of TLX/NR2E1 That Triggers Hippocampal Neurogenesis. Proceedings of the National Academy of Sciences 2022, 119 (13), e2023784119.
(85) Hart, T.; Chandrashekhar, M.; Aregger, M.; Steinhart, Z.; Brown, K. R.; MacLeod, G.; Mis, M.; Zimmermann, M.; Fradet-Turcotte, A.; Sun, S.; Mero, P.; Dirks, P.; Sidhu, S.; Roth, F. P.; Rissland, O. S.; Durocher, D.; Angers, S.; Moffat, J. High-Resolution CRISPR Screens Reveal Fitness Genes and Genotype-Specific Cancer Liabilities. Cell 2015, 163 (6), 1515-1526.
(86) Chen, E. Y.; Tan, C. M.; Kou, Y.; Duan, Q.; Wang, Z.; Meirelles, G. V.; Clark, N. R.; Ma'ayan, A. Enrichr: Interactive and Collaborative HTML5 Gene List Enrichment Analysis Tool. BMC Bioinformatics 2013, 14 (1), 128.
(87) Kuleshov, M. V.; Jones, M. R.; Rouillard, A. D.; Fernandez, N. F.; Duan, Q.; Wang, Z.; Koplev, S.; Jenkins, S. L.; Jagodnik, K. M.; Lachmann, A.; McDermott, M. G.; Monteiro, C. D.; Gundersen, G. W.; Ma'ayan, A. Enrichr: A Comprehensive Gene Set Enrichment Analysis Web Server 2016 Update. Nucleic Acids Res 2016, 44 (W1), W90-W97.
(88) Xie, Z.; Bailey, A.; Kuleshov, M. V.; Clarke, D. J. B.; Evangelista, J. E.; Jenkins, S. L.; Lachmann, A.; Wojciechowicz, M. L.; Kropiwnicki, E.; Jagodnik, K. M.; Jeon, M.; Ma'ayan, A. Gene Set Knowledge Discovery with Enrichr. Current Protocols 2021, 1 (3), e90.
(89) Kanehisa, M. KEGG: Kyoto Encyclopedia of Genes and Genomes. Nucleic Acids Research 2000, 28 (1), 27-30. (90) Subramanian, A.; Tamayo, P.; Mootha, V. K.; Mukherjee, S.; Ebert, B. L.; Gillette, M. A.; Paulovich, A.; Pomeroy, S. L.; Golub, T. R.; Lander, E. S.; Mesirov, J. P. Gene Set Enrichment Analysis: A Knowledge-Based Approach for Interpreting Genome-Wide Expression Profiles. Proc Natl Acad Sci USA 2005, 102 (43), 15545-15550. 2.
(91) Liberzon, A.; Subramanian, A.; Pinchback, R.; Thorvaldsdóttir, H.; Tamayo, P.; Mesirov, J. P. Molecular Signatures Database (MSigDB) 3.0. Bioinformatics 2011, 27 (12), 1739-1740.

1. A method of treating medulloblastoma (MB) comprising administering a therapeutically effective amount of a stearoyl-CoA Desaturase 1 (SCD1) inhibitor to a subject in need thereof.

2. The method of claim 1, wherein the medulloblastoma is high-risk medulloblastoma.

3. The method of claim 2, wherein the high-risk medulloblastoma comprises a medulloblastoma in a subject that is less than three years old, a medulloblastoma that has metastasized, a residual medulloblastoma from incomplete resection with a residual amount of tumour >1.5 cm², large cell/anaplastic (LCA) medulloblastoma, a recurrent medulloblastoma, medulloblastoma with amplification of MYC or MYCN, a Group 3 (G3) medulloblastoma (G3-MB), or a Group 4 (G4) medulloblastoma (G4-MB), or a combination thereof

4. The method of claim 2, wherein the high-risk medulloblastoma comprises a G3-MB, a G4-MB, G3 medulloblastoma that is MYC amplified (MYC-driven G3-MB), or a G4 medulloblastoma that is MYC amplified (MYC-driven G4-MB).

5. The method of claim 1, wherein treating medulloblastoma comprises inhibiting de novo lipid synthesis.

6. The method of claim 1, wherein treating medulloblastoma comprises inhibiting SCD1.

7. The method of claim 1, wherein the “subject in need thereof” is a subject having medulloblastoma or a subject that has had medulloblastoma.

8. The method of claim 1, wherein the SCD1 inhibitor is administered as soon as possible after medulloblastoma diagnosis.

9. The method of claim 1, wherein the SCD1 inhibitor is any agent that inhibits expression of SCD1 gene or protein, that induces SCD1 protein degradation or that inhibits SCD1 protein activity.

10. The method of claim 1, wherein the SCD1 inhibitor is a small molecule inhibitor of SCD1 protein activity, or a pharmaceutically acceptable salt, solvate and/or prodrug thereof.

11. The method of claim 10, wherein the small molecule inhibitor of SCD1 protein activity or a pharmaceutically acceptable salt, solvate and/or prodrug thereof is selected from A939572, MF-438, CVT-11127, CVT-12012, CAY10566, T-3764518, BZ36, SSI-4, SW208108, SW203668 and YTX-7739.

12. The method of claim 10, wherein the small molecule inhibitor of SCD1 protein activity is CAY10566.

13. The method of claim 1, wherein the method comprises administering a therapeutically effective amount of a SC1 inhibitor in combination with another known agent useful for treating medulloblastoma in a subject in need thereof.

14. The method of claim 13, wherein the another known agent useful for treating medulloblastoma is an agent for inhibiting de novo lipid synthesis.

15. The method of claim 13, wherein the another known agent useful for treating medulloblastoma is an inhibitor of lipid metabolism,

16. The method of claim 13, wherein the another known agent useful for treating medulloblastoma is inhibitor of glycine metabolism, serine metabolism, or threonine metabolism.

17. The method of claim 16, wherein the another known agent useful for treating medulloblastoma is an inhibitor of serine metabolism.

18. The method of claim 13, wherein the another known agent useful for treating medulloblastoma is an inhibitor of phosphoglycerate dehydrogenase (PHGDH).

19. The method of claim 13, wherein the another known agent useful for treating medulloblastoma is an agent useful for inhibiting pro-growth signaling cascades, including mitogen-induced kinases (MAPK, ERK1/2), JNK, and Akt, β-catenin, GSK3b, or JNK.

20. A pharmaceutical composition comprising an effective amount of a SCD1 inhibitor and a pharmaceutically acceptable carrier wherein the SCD1 inhibitor is present in amount effective to treat medulloblastoma.
